SEQUENCE LISTING (2 sequences)

SEQ ID NO: 1 - protein, 10 aa, Homo sapiens
YNWNSFGLRF

SEQ ID NO: 2 - protein, 10 aa, synthetic construct
  MOD_RES 1: note = N-Acetyl-3-(2-naphthyl)-D-alanine
  MOD_RES 2: note = 4-Chloro-D-phenylalanine
  MOD_RES 3: note = 3-(3-Pyridyl)-D-alanine
  MOD_RES 6: note = D-Citrulline
  MOD_RES 10: note = D-Alanine
AFASYRLRPA

(19) United States
(12) Patent Application Publication
(10) Pub. No.: US 2026/0034192 A1
(43) Pub. Date: Feb. 5, 2026

(54) USE OF GNRH ANTAGONISTS IN MAMMALS TO SYNCHRONIZE FOLLICULAR WAVE EMERGENCE

(71) Applicant: University of Saskatchewan, Saskatoon (CA)

(72) Inventors: Jaswant Singh, Saskatoon (CA); Gregg P. Adams, Saskatoon (CA); Carlos Eduardo Leonardi, Santa Maria (BR); Muhammad Anzar, Saskatoon (CA)

(21) Appl. No.: 18/996,649

(22) PCT Filed: Jul. 17, 2023
(86) PCT No.: PCT/CA2023/050960

§ 371 Date: Jan. 17, 2025

Related U.S. Application Data
(60) Provisional application No. 63/390,080, filed on Jul. 18, 2022.

(51) Int. Cl. A61K 38/09; A61K 31/4184; A61K 31/513; A61K 31/519; A61K 31/5575; A61K 31/57; A61P 15/08

(52) U.S. Cl. CPC A61K 38/09; A61K 31/4184; A61K 31/513; A61K 31/519; A61K 31/5575; A61K 31/57; A61P 15/08

(57) ABSTRACT

The present disclosure relates to methods for reproductive management of mammalian animals using a GnRH antagonist such as Cetrorelix. Specifically, the GnRH antagonist can be used for synchronizing follicular wave emergence in a population of female mammals, and/or for fixed-time reproductive management protocols such as oocyte collection protocols, embryo collection protocols, artificial insemination protocols or embryo transfer protocols. Also described are devices and kits for reproductive management, comprising a GnRH antagonist and optionally one or more additional drugs useful for reproductive management.

### CROSS-REFERENCE TO RELATED APPLICATIONS

This application claims the benefit of priority to U.S. Provisional Application No. 63/390,080, filed Jul. 18, 2022, the contents of which are incorporated herein by reference in their entirety.

### INCORPORATION OF SEQUENCE LISTING

A computer readable form of the Sequence Listing “13764-P65604PC00_SequenceListing.XML” (4,452 bytes) created on Jul. 17, 2023, is herein incorporated by reference.

### FIELD

The present disclosure relates generally to methods, uses, devices and kits for reproductive management in mammals using a GnRH antagonist. In particular, the present disclosure relates to the use of a GnRH antagonist for synchronizing follicular wave emergence (FWE) in mammals. The methods, uses, devices and kits have applications in ovulation synchronization, fixed-time artificial insemination protocols and other reproductive management strategies.

### INTRODUCTION

Existing animal breeding management protocols for fixed-time artificial insemination as well as donor/recipient management for embryo transfer (primarily, cattle and sheep) incorporate synchronization of emergence of a new follicular wave as the fundamental reset point. This concept is not clinically practiced in humans. Methods currently used to synchronize ovarian follicular wave emergence (FWE) in animals include the use of estradiol, gonadotropin-releasing hormone/luteinizing hormone (GnRH/LH), prostaglandin (PGF), or ultrasound-guided ablation of the dominant follicle, or combinations of these various treatments. Estradiol is the most effective drug, and a single-injection treatment is used widely in South American countries but has been banned by the European Union for reproductive management in food animals. Estradiol is not preferred in the rest of the World (including USA and Canada) due to perceived environmental concerns around estrogenic compounds. Single treatment with GnRH/LH and/or prostaglandin (either as monotherapy, or as a combination of a single GnRH/LH injection in combination with a single prostaglandin injection) is not as effective due to dependence on eliciting ovulation. Combination protocols, such as GnRH+ progesterone controlled internal drug release (CIDR)-PGF-GnRH (e.g. Ovsynch or co-synch protocols), and multi-dose protocols (e.g. two injections of PGF) often involve multiple treatments at 7- or 10-day intervals. Follicle ablation is the most effective method for inducing FWE but requires expensive equipment and technical skills, and is not practical at the herd level. By a conservative estimate, more than 15 million cattle undergo artificial insemination in Brazil alone (one of the largest markets) annually.

Ultrasonographic studies in early 90's characterized the pattern of follicle growth in cattle and since then the bovine model has been used extensively to understand the patterns in other mammalian species including humans. Follicular growth in cattle occurs in a wave-like pattern with the majority of the estrous cycles comprising two to three waves. In cattle, each follicular wave is characterized by the growth of between 8 and 41 small follicles (2-4 mm in diameter). A surge of follicle-stimulating hormone (FSH) precedes the emergence of each wave. At the beginning of a wave, all follicles require FSH for growth; therefore, emergence of follicular waves are temporally associated with circulating FSH levels in cattle and other mammals. Bovine follicles grow at about the same rate for approximately 2 days, then one follicle is “selected” to become the dominant follicle while the rest stop growing and regress (Ginther et al., 1989; Knopf et al., 1989). The selection of a dominant follicle (at ≅8 mm size) is associated with declining levels of FSH in circulation (Adams et al., 1992) due to feed back to the hypothalamus and pituitary from the follicular products (Adams and Singh, 2021). All follicles in a wave have growing (increasing diameter), static (constant diameter) and regressing (decreasing diameter) phases. The presumptive dominant follicle differentially develops luteinizing hormone (LH) receptor expression close to the time of selection (3 days after wave emergence in cattle) while subordinate follicles have no or low LH receptors. FSH plasma levels decrease between wave emergence and the time of selection of the dominant follicle. Studies have shown that the granulosa cells of the bovine dominant follicle acquire LH receptors around the time of selection, hence, the dominant follicle continues to grow by LH support in the face of an FSH nadir (Adams and Singh, 2021). The dominant follicle produces substances (mostly estradiol) that keeps FSH low and prevents emergence of the next wave. Once the dominant follicle regresses or ovulates, follicular products (e.g., estradiol) decrease to levels low enough to allow circulating concentrations of FSH to surge, resulting in a new wave emergence.

Cattle and camelids release one egg/oocyte at the time of ovulation (that is, both are monovular mammals) and give birth to a single offspring. Sheep and goats are multiovular species, giving birth to 2 to 3 kids/lambs. Control mechanisms for wave emergence and follicular growth are similar between cows, sheep, goats, and camelids. However, in contrast to the other ruminant species, camelids are classified as induced ovulators whereby copulatory stimulation is necessary for ovulation to take place (San Martin et al., 1968; England et al., 1969; Fernandez-Baca et al., 1970a). The presence of a functional dominant follicle plays a key role in the ability of camelids to be receptive to mating and to ovulate (Adams et al., 1990; Bravo et al., 1991; Vaughan et al., 2004). If copulation (and ensuing ovulation) does not occur, dominant follicular development continues to take place in a wave-like pattern that is characterized by a periodic increase in the number of follicles every 20 to 25 days (inter-wave interval) and selection of the dominant follicle followed by regression (Adams et al., 1990; Vaughan et al., 2004). In order to simplify herd management practices, the ovulations of a group of llamas could be synchronized for artificial insemination by the induction of a new follicular wave. Hormonal studies conducted in camelids have attempted to induce ovulation with the use of GnRH (Bravo et al., 1992; Aller et al., 1999; Cancino et al., 1999; Huanca et al., 2001), LH (Huanca et al., 2001; Taylor et al., 2000) and hCG (Adam et al., 1992; Bourke et al., 1992; Correa et al., 1997; Ratto et al., 1997) however, the results remain variable.

Both selection of the dominant follicle and ovulation require hormonal stimulation from FSH and LH (Adams et al., 2008). FSH and LH are both released from the anterior pituitary following GnRH secretion from the hypothalamus. In human assisted reproduction practices, endogenous GnRH membrane receptors (located in the pituitary) are commonly inhibited by GnRH antagonists such as Cetrorelix (Kovacs et al., 2001). Cetrorelix (trade name: Cetrotide™) has been utilized as a fertility treatment for women in order to avoid premature increases in LH during ovarian stimulation (EMD Serono Inc., 2008). Its action to prevent the ovulation of existing follicle(s) is well-known and thus clinically used in human FSH stimulation protocols for assisted reproduction. In such protocols, Cetrorelix is administered at the same time as FSH to block ovulation of FSH-induced follicles.

Cetrorelix is a synthetic decapeptide (Acetyl-D-3-(2′-naphtyl)-alanine-D-4-chlorophenylalanine-D-3-(3′-pyridyl)-alanine-L-serine-L-tyrosine-D-citruline-L-leucine-L-arginine-L-proline-D-alanine-amide; SEQ ID NO: 2) that acts as a GnRH/LHRH antagonist. Cetrorelix occupies GnRH receptors on gonadotophs in adenohypophysis, thereby preventing release of LH even in the presence of GnRH pulses. In human assisted reproduction protocols, Cetrorelix is used as a daily subcutaneous (s/c) injection of 0.25 mg for up to 6 days, or as an initial s/c bolus of 3 mg followed by daily doses of 0.25 mg after first 4 days. Its elimination half-life is rather short (5 hr after single injection of 0.25 mg in women).

Cetrorelix is known to lower the circulating concentration of LH, suppress the growth of existing single dominant follicle or multiple large follicles (e.g. during FSH stimulation of the ovaries) and to prevent pre-mature ovulation. Cetrorelix is used clinically in assisted reproductive technologies for women (e.g. to prevent pre-mature ovulations during FSH stimulation in human IVF cycles). Other GnRH antagonists are also used for this purpose in assisted reproduction in humans. During human FSH stimulation protocols, multiple follicles grow to ovulatory size, and cetrorelix is co-administered with FSH to prevent ovulation of existing multiple dominant follicles at the time of treatment.

The effects of GnRH antagonists such as Cetrorelix have been studied in cattle and other domestic animals.

Nivet et al (2018) investigated the mechanisms behind oocyte competence when LH was inhibited by Cetrorelix. Cetrorelix treatment was given for 4 days at the end of superstimulation in heifers. A significant reduction in the number of follicles at >10 mm diameter was observed during the dominant stage of development.

Haughian et al (2013) describe the effects of the GnRH antagonist acyline on follicle development. Acyline was given just before ovulation and multiple injections continued after ovulation and emergence of the follicular wave. Acyline did not reduce the concentrations of FSH during the periovulatory surge and early follicle development was unaffected, although subsequent growth of a dominant follicle (>8.0 mm) was prevented by acyline. Failure to select a dominant follicle in the acyline group was associated with reduced concentrations of LH but not FSH. Maximum diameter of dominant follicle in control animals (13.3 mm) was greater than in acyline-treated animals (7.7 mm).

Ginther et al (2012) describe the effects of acyline on follicle development. Acyline was given on post-ovulation Day 15, 16 and 17 to heifers. It caused the regression of Wave 2 dominant follicle and emergence of Wave 3. The heifers that were treated with acyline had an interovulatory interval of 25 to 34 days compared to 19 to 23 days in the non-treated heifers. Synchronization of wave emergence was not examined or discussed.

Silva et al (2011) describe the effects of Cetrorelix on follicle development in lamas. Cetrorelix inhibited the preovulatory LH surge and ovulation induced by GnRH and ovulation inhibiting factor (OIF) in llamas.

Ulker et al (2001) reports that Cetrorelix suppresses LH and progesterone secretion in cattle without affecting estrous cycle length.

Oussaid et al (2000) describe the effects of the GnRH antagonist antarelix on follicle development. Treatment of superstimulated heifers with antarelix for 36-60 hours led to follicular atresia without effecting on oocyte competence as determined by in vitro embryo production.

Fike et al (1997) describe the effects of the LHRH antagonist SB-75 on follicle development. Treatment of animals with SB-75 from Day 2 to 12 of the oestrous cycle resulted in modulation in circulating LH and FSH and led to suppressed dominant follicle persistence and oestradiol secretion. The effects of SB-75 on dominant follicle sizes and follicle number are described.

Madill et al (1994) describe the effects of SB-75 on hormone levels and ovulation SB-75 can be used to delay LH surge (detected analysis of blood samples by radioimmunoassay) and ovulation (detected by ultrasound) in superovulated heifers.

Leonardi (2018) utilized a GnRH antagonist experimentally in cattle to test the effects of kisspeptin. The study involved the administration of Cetrorelix on Day 6 of follicular development when plasma progesterone concentrations were low.

### SUMMARY

There is a need to develop new methods for the manipulation of follicular waves in large and small ruminants, camelids, and other livestock that are both effective and efficient when implemented under field conditions. As described herein, the inventors have demonstrated that administration of the GnRH antagonist Cetrorelix results in the emergence of a new follicular wave within a consistent time-frame in female mammals such as cattle and alpacas. Accordingly, the present disclosure relates to the use of a GnRH antagonist for the reproductive management of female mammals. The methods and uses described herein are non-therapeutic.

An aspect includes a non-therapeutic method of synchronizing follicular wave emergence (FWE) in a population of female mammals, the method comprising administering an effective amount of a GnRH antagonist to each mammal of the population of female mammals.

In an embodiment, the GnRH antagonist comprises Cetrorelix, acyline, antarelix/teverelix, degarelix, ganirelix, antide, relugolix, elagolix, abarelix, prazarelix, ramorelix, antide, detirelix, ozarelix, linzagolix, opigolix, sufugolix and A-75998. In an embodiment, the GnRH antagonist comprises abarelix. In an embodiment, the GnRH antagonist comprises degarelix. In an embodiment, the GnRH antagonist comprises relugolix. In an embodiment, the GnRH antagonist comprises Cetrorelix.

In an embodiment, the GnRH antagonist is administered by intramuscular injection or subcutaneous injection.

In an embodiment, the administering the GnRH antagonist comprises at least one treatment, optionally 2 or 3 treatments.

In an embodiment, the GnRH antagonist comprises Cetrorelix delivered by intramuscular injection at a dose of about 2.5 ug/kg body weight to about 40 ug/kg body weight, optionally at a dose of about 5 ug/kg, 7.5 ug/kg, 10 ug/kg, or 20 ug/kg body weight.

In an embodiment, the GnRH antagonist is administered by vaginal drug releasing device.

In an embodiment, the method further comprises co-administering progesterone or a progesterone analog, optionally the progesterone or progesterone analog is administered by vaginal drug releasing device.

In an embodiment, the mammals are domestic livestock, optionally the domestic livestock are bovine, camelid, equine, porcine, ovine or caprine species.

In an embodiment, the mammals are pets and wildlife animals, optionally bison, elk, caribou, deer, muskox, non-human primates, canines, or felines, optionally tigers, lions, or panthers.

In an embodiment, the mammals are humans.

In an embodiment, the population is a mixed population.

In an embodiment, the method further comprises inducing ovulation in each mammal of the population of female mammals at a fixed time following administration of the GnRH antagonist, comprising
I) administering an effective amount of an ovulation inducing agent at a fixed time following the administration of the GnRH antagonist; or
II) co-administering an effective amount of progesterone or progesterone analog with the GnRH antagonist; withdrawing the progesterone or progesterone analog and administering an effective amount of prostaglandin (PGF) or a PGF analog at a fixed time following the administration of the GnRH antagonist; and administering an effective amount of an ovulation inducing agent at a fixed time following the withdrawal of the progesterone or progesterone analog and/or administration of the PGF or PGF analog.

In an embodiment, the method further comprises artificially inseminating each mammal of the population of female mammals at a fixed time following administration of the ovulation inducing agent.

In an embodiment, the method further comprises mating each mammal of the population of female mammals at a fixed time following administration of the ovulation inducing agent.

In an embodiment, the method further comprises transferring an embryo to each animal of the population of female mammals at a fixed time following administration of the ovulation inducing agent.

Another aspect includes a non-therapeutic method of synchronizing ovulation in a population of female mammals, the method comprising:
I) a) administering an effective amount of a GnRH antagonist; and b) administering an effective amount of an ovulation inducing agent at a fixed time following the administration of the GnRH antagonist, to each mammal of the population of female mammals; or
II) a) co-administering an effective amount of a GnRH antagonist and an effective amount of progesterone or a progesterone analog; b) withdrawing the progesterone or progesterone analog and administering an effective amount of prostaglandin (PGF) or a PGF analog at a fixed time following the administration of the GnRH antagonist; and c) administering an effective amount of an ovulation inducing agent at a fixed time following the withdrawal of the progesterone or progesterone analog and/or administration of the PGF or PGF analog, to each mammal of the population of female mammals.

Another aspect includes a non-therapeutic method of initiating follicular wave emergence in a female mammal, the method comprising: administering an effective amount of a GnRH antagonist to the female mammal.

Another aspect includes a non-therapeutic method of inducing ovulation in a female mammal, the method comprising:
I) a) administering an effective amount of a GnRH antagonist to the female mammal; and b) administering an effective amount of an ovulation inducing agent at a fixed time following the administration of the GnRH antagonist; or
II) a) co-administering an effective amount of a GnRH antagonist and an effective amount of progesterone or a progesterone analog to the female mammal; b) withdrawing the progesterone or progesterone analog and administering an effective amount of prostaglandin (PGF) or a PGF analog at a fixed time following the administration of the GnRH antagonist; and c) administering an effective amount of an ovulation inducing agent at a fixed time following the withdrawal of the progesterone or progesterone analog and/or administration of the PGF or PGF analog.

Another aspect includes a non-therapeutic fixed-time embryo transfer method, comprising:
I) a) administering an effective amount of a GnRH antagonist to a female mammal; b) administering an effective amount of an ovulation inducing agent at a fixed time following the administration of the GnRH antagonist; and c) transferring one or more embryos to the female mammal at a fixed time following the administration of the ovulation inducing agent; or
II) a) co-administering an effective amount of a GnRH antagonist and an effective amount of progesterone or a progesterone analog to a female mammal; b) withdrawing the progesterone or progesterone analog and administering an effective amount of prostaglandin (PGF) or a PGF analog at a fixed time following the administration of the GnRH antagonist; c) administering an effective amount of an ovulation inducing agent at a fixed time following the withdrawal of the progesterone or progesterone analog and/or administration of the PGF or PGF analog; and d) transferring one or more embryos to the female mammal at a fixed time following the administration of the ovulation inducing agent.

Another aspect includes a non-therapeutic fixed-time superstimulatory or superovulatory method, the method comprising:
I) a) administering an effective amount of a GnRH antagonist to a female mammal; b) administering an effective amount of FSH or an FSH agonist to the female mammal at a fixed time following the administration of the GnRH antagonist, provided that the FSH or FSH agonist is not co-administered with the GnRH antagonist; and c) administering an effective amount of an ovulation inducing agent at a fixed time following the administration of the FSH or FSH analog; or
II) a) co-administering an effective amount of a GnRH antagonist and an effective amount of progesterone or a progesterone analog to a female mammal; b) administering an effective amount of FSH or an FSH agonist to the female mammal at a fixed time following the administration of the GnRH antagonist, provided that the FSH or FSH agonist is not co-administered with the GnRH antagonist; c) withdrawing the progesterone or progesterone analog and administering an effective amount of prostaglandin (PGF) or a PGF analog at a fixed time following the administration of the FSH or FSH agonist; and d) administering an effective amount of an ovulation inducing agent at a fixed time following the withdrawal of the progesterone or progesterone analog and/or administration of the PGF or PGF analog.

In an embodiment, the method further comprises collecting one or more oocytes at a fixed time following the administration of the ovulation inducing agent. In an embodiment, the method further comprises I) d) or II) e) artificially inseminating the female mammal at a fixed time following the administration of the ovulation inducing agent; and I) e) or II) f) collecting one or more embryos at a fixed time following the artificial insemination. In an embodiment, the method further comprises I) d or II) e) mating the female mammal at a fixed time following the administration of the ovulation inducing agent; and I) e) or II) f) collecting one or more embryos at a fixed time following the artificial insemination.

Another aspect includes a non-therapeutic fixed-time embryo collection method, the method comprising:
I) a) administering an effective amount of a GnRH antagonist to a female mammal; b) administering an effective amount of FSH or an FSH agonist to the female mammal at a fixed time following the administration of the GnRH antagonist, provided that the FSH or FSH agonist is not co-administered with the GnRH antagonist; c) administering an effective amount of an ovulation inducing agent at a fixed time following the administration of the FSH or FSH analog; d) artificially inseminating or mating the female mammal at a fixed time following the administration of the ovulation inducing agent; and e) collecting one or more embryos at a fixed time following the artificial insemination or mating; or
II) a) co-administering an effective amount of a GnRH antagonist and an effective amount of progesterone or a progesterone analog to a female mammal; b) administering an effective amount of FSH or an FSH agonist to the female mammal at a fixed time following the administration of the GnRH antagonist, provided that the FSH or FSH agonist is not co-administered with the GnRH antagonist; c) withdrawing the progesterone or progesterone analog and administering an effective amount of prostaglandin (PGF) or a PGF analog at a fixed time following the administration of the FSH or FSH agonist; d) administering an effective amount of an ovulation inducing agent at a fixed time following the withdrawal of the progesterone or progesterone analog and/or administration of the PGF or PGF analog; e) artificially inseminating or mating the female mammal at a fixed time following the administration of the ovulation inducing agent; and f) collecting one or more embryos at a fixed time following the artificial insemination or mating.

Another aspect includes a non-therapeutic fixed-time oocyte collection method, the method comprising:
I) a) administering an effective amount of a GnRH antagonist; and b) collecting one or more oocytes at a fixed time following the administration of the GnRH antagonist;
II) a) administering an effective amount of a GnRH antagonist to a female mammal; b) administering an effective amount of FSH or an FSH agonist to the female mammal at a fixed time following the administration of the GnRH antagonist, provided that the FSH or FSH agonist is not co-administered with the GnRH antagonist; and c) collecting one or more oocytes at a fixed time following the administration of the FSH or FSH agonist;
III) a) administering an effective amount of a GnRH antagonist to a female mammal; b) administering an effective amount of FSH or an FSH agonist to the female mammal at a fixed time following the administration of the GnRH antagonist, provided that the FSH or FSH agonist is not co-administered with the GnRH antagonist; c) administering an effective amount of an ovulation inducing agent at a fixed time following the administration of the FSH or FSH analog; and d) collecting one or more oocytes at a fixed time following the administration of the ovulation inducing agent; or
IV) a) co-administering an effective amount of a GnRH antagonist and an effective amount of progesterone or a progesterone analog to a female mammal; b) administering an effective amount of FSH or an FSH agonist to the female mammal at a fixed time following the administration of the GnRH antagonist, provided that the FSH or FSH agonist is not co-administered with the GnRH antagonist; c) withdrawing the progesterone or progesterone analog and administering an effective amount of prostaglandin (PGF) or a PGF analog at a fixed time following the administration of the FSH or FSH agonist; d) administering an effective amount of an ovulation inducing agent at a fixed time following the withdrawal of the progesterone or progesterone analog and/or administration of the PGF or PGF analog; and e) collecting one or more oocytes at a fixed time following the administration of the ovulation inducing agent.

Another aspect includes a non-therapeutic fixed-time artificial insemination method, the method comprising:
I) a) administering an effective amount of a GnRH antagonist to a female mammal; b) administering an effective amount of an ovulation inducing agent at a fixed time following the administration of the GnRH antagonist; and c) artificially inseminating the female mammal at a fixed time following the administration of the ovulation inducing agent; or
II) a) co-administering an effective amount of a GnRH antagonist and an effective amount of progesterone or a progesterone analog to a female mammal; b) withdrawing the progesterone or progesterone analog and administering an effective amount of prostaglandin (PGF) or a PGF analog at a fixed time following the administration of the GnRH antagonist; c) administering an effective amount of an ovulation inducing agent at a fixed time following the withdrawal of the progesterone or progesterone analog and/or administration of the PGF or PGF analog; and d) artificially inseminating the female mammal at a fixed time following the administration of the ovulation inducing agent.

Another aspect includes a breeding management method, the method comprising:
I) a) administering an effective amount of a GnRH antagonist to a female mammal; b) administering an effective amount of an ovulation inducing agent at a fixed time following the administration of the GnRH antagonist; and c) mating the female mammal at a fixed time following the administration of ovulation inducing agent; or
II) a) co-administering an effective amount of a GnRH antagonist and an effective amount of progesterone or a progesterone analog to a female mammal; b) withdrawing the progesterone or progesterone analog and administering an effective amount of prostaglandin (PGF) or a PGF analog at a fixed time following the administration of the GnRH antagonist; c) administering an effective amount of an ovulation inducing agent at a fixed time following the withdrawal of the progesterone or progesterone analog and/or administration of the PGF or PGF analog; and d) mating the female mammal at a fixed time following the administration of the ovulation inducing agent.

In an embodiment of the aspects described herein, the GnRH antagonist comprises Cetrorelix, abarelix, degarelix, ganirelix, antarelix/teverelix, prazarelix, ramorelix, antide, detirelix, ozarelix, acyline, elagolix, linzagolix, relugolix, opigolix, sufugolix or A-75998. In an embodiment, the GnRH antagonist comprises abarelix. In an embodiment, the GnRH antagonist comprises degarelix. In an embodiment, the GnRH antagonist comprises relugolix. In an embodiment, the GnRH antagonist comprises Cetrorelix.

In an embodiment of the aspects described herein, the GnRH antagonist is administered by intramuscular injection or subcutaneous injection.

In an embodiment of the aspects described herein, the administering the GnRH antagonist comprises at least one treatment, optionally 2 or 3 treatments.

In an embodiment of the aspects described herein, the GnRH antagonist comprises Cetrorelix delivered by intramuscular injection at a dose of about 5 ug/kg body weight to about 40 ug/kg body weight, optionally at a dose of about 20 ug/kg body weight.

In an embodiment of the aspects described herein, the GnRH antagonist is administered by vaginal drug releasing device.

In an embodiment of the aspects described herein, the mammals are domestic livestock, optionally the domestic livestock are bovine, camelid, equine, porcine, ovine or caprine. In an embodiment, the mammal is a bovine.

In an embodiment of the aspects described herein, the mammals are wildlife animals, optionally bison, elk, caribou, deer, muskox, non-human primates, canines, or felines, optionally tigers, lions, or panthers.

In an embodiment of the aspects described herein, the mammal is a human.

In an embodiment of the aspects described herein, the method further comprises co-administering progesterone, optionally wherein the progesterone is administered by vaginal drug releasing device. In an embodiment, the progesterone drug releasing is administered for 7-8 days.

In an embodiment of the aspects described herein, the GnRH antagonist is administered on day 0 (D0), and the method further comprises inserting a progesterone drug releasing device on D0; administering the effective amount of PGF or PGF analog and removing the progesterone drug releasing device on day 8 (D8); and administering the effective amount of GnRH on day 10 (D10).

In an embodiment of the aspects described herein, the PGF or PGF analog comprises prostaglandin 2 alpha (PGF2a), cloprostenol, dinoprost, dinoprost tromethamine, bimatoprost, travoprost, carboprost and latanoprost. In an embodiment, the PGF or PGF analog comprises PGF2a.

In an embodiment of the aspects described herein, the ovulation inducing agent comprises GnRH, LH, or estradiol, or an agonist, analog, ester, conjugate or recombinant product of any thereof. In an embodiment, the GnRH or GnRH analog or agonist comprises GnRH, buserelin, deslorelin, fertirelin, gonadorelin, goserelin, leuprorelin, or triptorelin. In an embodiment, the LH or LH analog, agonist, conjugate, or recombinant product comprises LH, human chroiogondotropin/hCG, or equine chriogonadotropin/eCG. In an embodiment, the estradiol or estradiol ester, analog, or agonist comprises estradiol-17beta, estradiol benzoate, estradiol valerate, or estradiol cypionate.

Another aspect includes a drug releasing device comprising a GnRH antagonist. In an embodiment, the GnRH antagonist comprises Cetrorelix, abarelix, degarelix, ganirelix, antarelix/teverelix, prazarelix, ramorelix, antide, detirelix, ozarelix, acyline, elagolix, linzagolix, relugolix, opigolix, sufugolix or A-75998. In an embodiment, the GnRH antagonist comprises abarelix. In an embodiment, the GnRH antagonist comprises degarelix. In an embodiment, the GnRH antagonist comprises relugolix. In an embodiment, GnRH antagonist is Cetrorelix.

In an embodiment, the drug releasing device provides controlled release of the GnRH antagonist over a period of about one day to about two days.

In an embodiment, the drug releasing device further comprises progesterone, wherein the drug releasing device provides controlled release of progesterone over a period of at least 5 days, at least 6 days, at least 7 days, at least 8 days, at least 9 days, at least 10 days, or for longer than 10 days.

In an embodiment, the drug releasing device is for use in synchronizing follicular wave emergence (FWE) in a population of female mammals, for use in synchronizing ovulation in a population of female mammals, for use in a fixed-time superstimulatory or superovulatory method, for use in a fixed-time embryo transfer method, for use in a fixed-time artificial insemination method, or for use in a fixed-time breeding method.

An aspect includes a non-therapeutic method of synchronizing follicular wave emergence in a population of female mammals, the method comprising inserting a drug releasing device comprising a GnRH antagonist as described herein into each mammal of the population of mammals.

The preceding section is provided by way of example only and is not intended to be limiting on the scope of the present disclosure and appended claims. Additional objects and advantages associated with the compositions and methods of the present disclosure will be appreciated by one of ordinary skill in the art in light of the instant claims, description, and examples. For example, the various aspects and embodiments of the disclosure may be utilized in numerous combinations, all of which are expressly contemplated by the present description. These additional advantages, objects, and embodiments are expressly included within the scope of the present disclosure. The publications and other materials used herein to illuminate the background of the disclosure, and in particular cases, to provide additional details respecting the practice, are incorporated by reference, and for convenience are listed in the appended reference section.

### DRAWINGS

Further objects, features and advantages of the disclosure will become apparent from the following detailed description taken in conjunction with the accompanying figures showing illustrative embodiments of the disclosure, in which:

FIG. 1 shows a schematic diagram of the experimental design used in Example 1. KP=Kisspeptin; Cetro=Cetrorelix.

FIG. 2 shows ovarian follicular dynamics and LH profiles in heifers treated in Example 1. A) Growth profile of dominant follicle in pubertal heifers treated at Day 6 of follicular wave (day of wave emergence=Day 0) with human Kisspeptin-10 given as three injections of 15 mg at 60 minutes interval over a 2-hour period (Kp10 group, n=5). Cetrorelix group (n=5) was injected GnRH antagonist (Cetrorelix, 20 μg/Kg body weight, intramuscular) 3 hours before kisspeptin treatment while the Control group (n=5) was given three intravenous injections of normal saline. B) Plasma LH plasma concentrations in Kp10, Cetrorelix and Control group heifers.

FIG. 3 shows a schematic diagram of the experimental design used in Example 2. WE=wave emergence; Cetro=Cetrorelix.

FIG. 4 shows a schematic timeline for Example 4. Cetrorelix (1.5 mg in 3 ml of 0.5 mg/ml in 5% D-Mannitol) per animal i/m daily was given on Day 1-2, Day 3-4, or Day 6-7 while first group served as untreated control (n=8 cattle per group). PGF=prostaglandin analog, Ov=ovulation, WE=Wave emergence=Day 0.

FIG. 5 shows dominant follicle growth pattern after Cetrorelix treatment. Cetro 1=Day 1-2 group, Cetro 3=Day 3-4 group, Cetro 6=Day 6-7 group.

FIG. 6 shows a schematic diagram of the experimental design for Example 5.

FIG. 7 shows dominant follicle growth pattern after a single Cetrorelix treatment.

FIG. 8 shows a schematic timeline of the experimental design for Example 6. Ov=ovulation, Cetro=cetrorelix, hCG=Human chorigondaotropin (Chorulon), D=Day, Cuemate=Intravaginal progesterone device, P4=progesterone, LH=luteinizing hormone, PGF=prostaglandin.

FIG. 9 shows a schematic diagram of the experimental design used in Example 7. PGF=prostaglandin F2alpha; AI=artificial insemination.

FIG. 10 shows the percentage of heifers showing estrous behavior/heat detection (left graph) and time of ovulation after CIDR removal/prostaglandin injection (right graph). D10, D11 and D12 refer to Day 10, 11 and 12, respectively, after Cetrorelix treatment. 48H, 72H and 96H refer to 48 hours, 72 hours and 96 hours after CIDR removal/PGF treatment in both panels.

FIG. 11 shows a schematic diagram of the experimental design used in Example 8. US=ultrasound; PGF=prostaglandin F2alpha; eCG=equine choriogonadotropin; AI=artificial insemination.

FIG. 12 shows LH levels (mIU/mL) secreted by pituitary cells after addition of 1 nM of GnRH. Cells were pretreated with cetrorelix, degarelix, relugolix and abarelix or not (control).

FIG. 13 shows LH levels (mIU/mL) secreted by pituitary cells after addition of 10 nM of GnRH. Cells were pretreated with cetrorelix, degarelix, relugolix and abarelix or not (control).

FIG. 14 shows plasma LH levels after GnRH-challenge in cattle pre-treated with GnRH antagonists. (A) shows bovine plasma LH levels (+SEM) after injection of cetrorelix. GnRH injection was given at Time 0 min: positive control (n=2) showed 1.5-fold increase in plasma LH concentration within 30 minutes while LH levels remained unchanged in heifers pre-treated with cetrorelix (n=2) 60 minutes before the GnRH injection (indicated by *). (B) shows the fold-change in plasma LH in animals pre-treated with Abarelix, Degarelix, Relugolix and Cetrorelix or control (no pre-treatment). Time 0 min=time of GnRH injection.

FIG. 15 shows a schematic diagram of the experimental design used in Example 11. PGF=prostaglandin F2alpha; FA=follicle ablation.

FIG. 16 shows a schematic diagram of the experimental design used in Example 12. PGF=prostaglandin F2alpha; FA=follicle ablation.

FIG. 17 shows a schematic diagram of the experimental design used in Example 13. PGF=prostaglandin F2alpha; FA=follicle ablation.

FIG. 18 shows a schematic diagram of the experimental design used in Example 14. PGF=prostaglandin F2alpha; AI=artificial insemination; OV=ovulation; Preg Check=pregnancy check; US=ultrasound.

### DESCRIPTION OF VARIOUS EMBODIMENTS

The following is a detailed description provided to aid those skilled in the art in practicing the present disclosure. Unless otherwise defined, all technical and scientific terms used herein have the same meaning as commonly understood by one of ordinary skill in the art to which this disclosure belongs. The terminology used in the description herein is for describing particular embodiments only and is not intended to be limiting of the disclosure. All publications, patent applications, patents, figures and other references mentioned herein are expressly incorporated by reference in their entirety.

Further, the definitions and embodiments described in particular sections are intended to be applicable to other embodiments herein described for which they are suitable as would be understood by a person skilled in the art. For example, in the following passages, different aspects of the disclosure are defined in more detail. Each aspect so defined may be combined with any other aspect or aspects unless clearly indicated to the contrary. In particular, any feature described herein may be combined with any other feature or features described herein.

The present disclosure relates to the non-therapeutic use of a GnRH antagonist for the reproductive management of mammals. As described herein, the inventors have demonstrated that administration of the GnRH antagonist Cetrorelix results in the synchronous emergence of a new follicular wave in female mammals such as cattle and alpacas. For example, the administration of a single Cetrorelix injection of 1.5 mg in an asynchronous population of alpacas resulted in the emergence of a new follicular wave on average 5.33 days later (variance of 7.38), compared to an average of 8.93 days (variance of 22.067) in control animals. The lower variance observed in the Cetrorelix-treated group (7.38 vs 22.067) indicates synchronous FWE emergence relative to the control. Because the mechanisms that control follicle dynamics are similar in all mammals, this synchronous FWE following the administration of a GnRH antagonist such as Cetrorelix is expected to be recapitulated in other mammalian species, albeit with species-specific temporal dynamics. Accordingly, Cetrorelix (and related GnRH receptor antagonists) can be used to manipulate the emergence of next follicular wave in female mammals including, for example, cattle, other domestic animals, wild mammals, non-human primates and in humans. The methods and uses described herein are non-therapeutic.

Use of Cetrorelix is known in humans to prevent premature ovulation of existing multiple dominant follicles in FSH stimulation IVF cycles. In contrast, the approach described herein allows a single injection or intravaginal application of the GnRH antagonist (for example as part of an intravaginal drug releasing device, optionally in combination with progesterone), to be used at any stage of the ovarian cycle to reset the emergence of a next (new) follicular wave at a consistent time from the time of treatment. Without wishing to be bound by theory, the synchronous emergence of follicular wave with Cetrorelix seems to rely on the fact that LH is consistently lowered at the same time in all animals. It may involve the same endocrine pathway as estradiol action on wave emergence but at different level (Estradiol prevents GnRH release while Cetrorelix blocks LH release). This “synchronization of ovarian follicular waves” is the basis of ovulation synchronization and fixed-time artificial insemination protocols, as well as gonadotropin-stimulation and superovulation treatment for embryo production/transfer in cattle and other domestic animals.

Single injection of Cetrorelix or other GnRH antagonist compounds is an alternative to the use of estradiol and/or GnRH for ovarian follicular wave synchronization in mammals (including humans) because it is non-steroidal, easy and cheap to synthesize, effective against all phases of the dominant follicle, and requires only a single treatment. Further, Cetrorelix or other GnRH antagonists can be impregnated in silicone or similar compounds as a quick-releasing drug to prepare intravaginal drug releasing devices, optionally in combination with progesterone, that will simplify treatment (single device without need for injection) and prevent second use of previously-used devices.

### I. Definitions

As used herein, the following terms may have meanings ascribed to them below, unless specified otherwise. However, it should be understood that other meanings that are known or understood by those having ordinary skill in the art are also possible, and within the scope of the present disclosure. All publications, patent applications, patents, and other references mentioned herein are incorporated by reference in their entirety. In the case of conflict, the present specification, including definitions, will control. In addition, the materials, methods, and examples are illustrative only and not intended to be limiting.

Where a range of values is provided, it is understood that each intervening value, to the tenth of the unit of the lower limit unless the context clearly dictates otherwise, between the upper and lower limit of that range and any other stated or intervening value in that stated range is encompassed within the description. Smaller ranges from any lower limit to any upper limit are contemplated. The upper and lower limits of these smaller ranges which may independently be included in the smaller ranges is also encompassed within the description, subject to any specifically excluded limit in the stated range. Where the stated range includes one or both of the limits, ranges excluding either or both of those included limits are also included in the description.

All numerical values within the detailed description and the claims herein are modified by “about” or “approximately” the indicated value, and take into account experimental error and variations that would be expected by a person having ordinary skill in the art. For example, “about” may mean plus or minus 10%, or plus or minus 5% of the indicated value to which reference is being made.

As used herein the singular forms “a”, “an”, and “the” include plural references unless the context clearly dictates otherwise.

The phrase “and/or,” as used herein, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified.

As used herein, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of” or, when used in the claims, “consisting of” will refer to the inclusion of exactly one element of a number or list of elements. In general, the term “or” as used herein shall only be interpreted as indicating exclusive alternatives (i.e., “one or the other but not both”) when preceded by terms of exclusivity, such as “either,” “one of,” “only one of,” or “exactly one of.”

As used herein, all transitional phrases such as “comprising,” “including,” “carrying,” “having,” “containing,” “involving,” “holding,” “composed of,” and the like are to be understood to be open-ended, i.e., to mean including but not limited to. Only the transitional phrases “consisting of” and “consisting essentially of” shall be closed or semi-closed transitional phrases, respectively

As used herein, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified.

It should also be understood that, in certain methods described herein that include more than one step or act, the order of the steps or acts of the method is not necessarily limited to the order in which the steps or acts of the method are recited unless the context indicates otherwise.

### II. Methods and Uses

As shown herein, the administration of the GnRH antagonist Cetrorelix at different stages of the ovarian cycle resulted in the emergence of a new follicular wave at a consistent time from the time of administration. Therefore, a GnRH antagonist, for example Cetrorelix, can be used to synchronize the emergence of a new follicular wave and optionally subsequent ovulation in a population of mammals, for example a herd of domestic livestock. This synchronous FWE is useful in reproductive management, for example, in inducing synchronous ovulation for fixed-time oocyte collection protocols, artificial insemination protocols, breeding, and/or embryo production/transfer, in domestic livestock. Accordingly, an aspect of the present disclosure is a non-therapeutic method of synchronizing follicular wave emergence (FWE) in a population of female mammals, the method comprising: administering an effective amount of a GnRH antagonist to each mammal of the population of female mammals. Another aspect of the disclosure includes a non-therapeutic method of synchronizing ovulation in a population of female mammals, the method comprising: administering an effective amount of a GnRH antagonist to each mammal of the population of female mammals; and administering an effective amount of an ovulation inducing agent at a fixed time following the administration of the GnRH antagonist. As will be understood in the art, in some cases, for example in species that are spontaneous ovulators (e.g. bovine, ovine, equine, porcine, canine), individual mammals in the population may have an existing corpus luteum which could interfere with synchronization. Accordingly, to circumvent the need to examine individual animals for the presence of an existing functional corpus luteum, additional treatments may be administered. For example, progesterone may be co-administered with the GnRH antagonist a) to prevent LH surge and ovulation, b) to prime uterine endometrium to prevent early PGF release after ovulation, and c) progesterone withdrawal sets up ovulatory cascade. PGF may be administered about the time of progesterone withdrawal to induce luteolysis of any existing corpus luteum. Accordingly, in an aspect, a non-therapeutic method of synchronizing ovulation in a population of mammals comprises: administering an effective amount of a GnRH antagonist and an effective amount of progesterone or a progesterone analog; withdrawing administration of the progesterone or progesterone analog and administering an effective amount of prostaglandin (PGF) or a PGF analog at a fixed time following the administration of the GnRH antagonist; and administering an effective amount of an ovulation inducing agent at a fixed time following the withdrawal of the progesterone or progesterone analog and/or administration of the PGF or PGF analog, to each mammal of the population of female mammals. The methods and uses described herein are non-therapeutic.

The GnRH antagonist may be administered at any stage of the ovulatory cycle. Accordingly, in various aspects and embodiments of the methods described herein, it is not necessary to know the stage of the cycle of the female mammal, and/or the population may be a mixed population.

In various embodiments of the aspects described herein, the method further comprises collecting one or more oocytes from each mammal, artificially inseminating each mammal, mating each mammal, collecting one or more embryos from each mammal, or transferring an embryo to each animal of the population of female mammals at a fixed time following administration of the ovulation inducing agent.

Another aspect described herein is a non-therapeutic method of initiating follicular wave emergence in a female mammal, the method comprising: administering an effective amount of a GnRH antagonist to the female mammal. A further aspect includes a non-therapeutic method of inducing ovulation in a female mammal, the method comprising: I) a) administering an effective amount of a GnRH antagonist to a female mammal; and b) administering an effective amount of an ovulation inducing agent at a fixed time following the administration of the GnRH antagonist; or II) a) co-administering an effective amount of a GnRH antagonist and an effective amount of progesterone or a progesterone analog to a female mammal; b) withdrawing administration of the progesterone or progesterone analog and administering an effective amount of prostaglandin (PGF) or a PGF analog at a fixed time following the administration of the GnRH antagonist; and c) administering an effective amount of an ovulation inducing agent at a fixed time following the withdrawal of the progesterone or progesterone analog and/or administration of the PGF or PGF analog.

Another aspect of the disclosure includes a non-therapeutic fixed-time artificial insemination method, the method comprising: I) a) administering an effective amount of a GnRH antagonist to a female mammal; b) administering an effective amount of an ovulation inducing agent at a fixed time following the administration of the GnRH antagonist; and c) artificially inseminating the female mammal at a fixed time following the administration of the ovulation inducing agent; or II) a) co-administering an effective amount of a GnRH antagonist and an effective amount of progesterone or a progesterone analog to a female mammal; b) withdrawing administration of the progesterone or progesterone analog and administering an effective amount of prostaglandin (PGF) or a PGF analog at a fixed time following the administration of the GnRH antagonist; c) administering an effective amount of an ovulation inducing agent at a fixed time following the withdrawal of the progesterone or progesterone analog and/or administration of the PGF or PGF analog; and d) artificially inseminating the female mammal at a fixed time following the administration of the ovulation inducing agent.

Another aspect of the disclosure includes a breeding management method, the method comprising: I) a) administering an effective amount of a GnRH antagonist to a female mammal; b) administering an effective amount of an ovulation inducing agent at a fixed time following the administration of the GnRH antagonist; and c) mating the female mammal following the administration of the ovulation inducing agent; or II) a) co-administering an effective amount of a GnRH antagonist and an effective amount of progesterone or a progesterone analog to a female mammal; b) withdrawing administration of the progesterone or progesterone analog and administering an effective amount of prostaglandin (PGF) or a PGF analog at a fixed time following the administration of the GnRH antagonist; c) administering an effective amount of an ovulation inducing agent at a fixed time following the withdrawal of the progesterone or progesterone analog and/or administration of the PGF or PGF analog; and d) mating the female mammal following the administration of the ovulation inducing agent. In these various aspects, it is not necessary to know the stage of the cycle of the female mammal, and/or the methods may be used on a mixed population of female mammals.

Another aspect of the disclosure includes a gonadotropin-stimulation and superovulation method, optionally for oocyte collection or embryo production/transfer, the method comprising: I) a) administering an effective amount of a GnRH antagonist to a female mammal; and b) administering an effective amount of FSH at a fixed time after GnRH antagonist administration; or II) a) co-administering an effective amount of a GnRH antagonist and an effective amount of progesterone or a progesterone analog to a female mammal; b) administering an effective amount of FSH at a fixed time after GnRH antagonist administration; and c) withdrawing administration of the progesterone or progesterone analog and administering an effective amount of prostaglandin (PGF) or a PGF analog at a fixed time following the administration of the FSH. In an embodiment, the method further comprises I) c) administering an effective amount of an ovulation inducing agent at a fixed time following the administration of the FSH; or II) d) administering an effective amount of an ovulation inducing agent at a fixed time following the withdrawal of the progesterone or progesterone analog and/or administration of the PGF or PGF analog. In an embodiment, administration of the GnRH antagonist, FSH, PGF or PGF analog, or ovulation inducing agent is followed by collection of multiple oocytes, for example for in vitro fertilization/embryo production. In an embodiment, administration of the GnRH antagonist, FSH, PGF or PGF analog, or ovulation inducing agent is followed by artificial insemination of animals, and collection of multiple embryos, optionally about 5 to 8 days after artificial insemination. In these various aspects and embodiments, it is not necessary to know the stage of the cycle of the female mammal, and/or the methods may be used on a mixed population of female mammals.

Also provided herein is a use of a GnRH antagonist for synchronizing follicular wave emergence and/or ovulation in a population of female mammals. Further provided herein is a use of a GnRH antagonist for initiating follicular wave emergence in a female mammal. Further provided herein is a use of a GnRH antagonist for inducing ovulation in a female mammal. Further provided herein is a use of a GnRH antagonist in a fixed time artificial insemination method. Further provided herein is a use of a GnRH antagonist in a gonadotropin-stimulation and superovulation treatment method, optionally for embryo production/transfer methods. In these aspects, it is not necessary to know the stage of the cycle of the female mammal, and/or the use may be in a mixed population of female mammals.

Also disclosed herein is the use of a GnRH antagonist, optionally Cetrorelix, in the manufacture of a medicament for synchronizing follicular wave emergence in a population of female mammals. An aspect includes the use of a GnRH antagonist, optionally Cetrorelix, in the manufacture of a medicament for synchronizing ovulation in a population of female mammals. An aspect includes a use of a GnRH antagonist in the manufacture of a medicament for initiating follicular wave emergence in a female mammal. An aspect includes a use of a GnRH antagonist in the manufacture of a medicament for inducing ovulation in a female mammal. An aspect includes use of a GnRH antagonist in the manufacture of a medicament for fixed-time artificial insemination methods, and/or for gonadotropin-stimulation and superovulation treatment for embryo production/transfer. In these aspects, it is not necessary to know the stage of the cycle of the female mammal, and/or the medicament may be for use in a mixed population of female mammals.

In a further aspect of the disclosure, the GnRH antagonist, optionally Cetrorelix, is for use in synchronizing follicular wave emergence in a population of female mammals. In one aspect, the GnRH antagonist is for use in synchronizing ovulation in a population of female mammals. In one aspect the GnRH antagonist is for use in initiating follicular wave emergence in a female mammal. In one aspect the GnRH antagonist is for use in inducing ovulation in a female mammal. In one aspect, the GnRH antagonist is for use in fixed-time artificial insemination methods. In another aspect, the GnRH antagonist is for use in gonadotropin-stimulation and superovulation treatment for embryo production/transfer. In these aspects, it is not necessary to know the stage of the cycle of the female mammal, and/or the GnRH antagonist may be for use in a mixed population of female mammals.

The term “GnRH antagonist” as used herein refers to an agent that reduces, decreases, or otherwise blocks activity of GnRH and/or GnRH receptors, and includes, without limitation, small molecules, peptides, and antibodies (and fragments thereof). GnRH antagonists are also referred to in the literature as luteinizing hormone releasing hormone (LHRH) antagonists. Commonly used GnRH antagonists include, without limitation, the peptides Cetrorelix, abarelix, degarelix, ganirelix, antarelix/teverelix, prazarelix, ramorelix, antide, detirelix, ozarelix, acyline and the small-molecule compounds elagolix, linzagolix, relugolix, opigolix, sufugolix and A-75998 (see also Table 24). Accordingly, in an embodiment, the GnRH antagonist is Cetrorelix, abarelix, degarelix, ganirelix, antarelix/teverelix, prazarelix, ramorelix, antide, detirelix, ozarelix, acyline, elagolix, linzagolix, relugolix, opigolix, sufugolix or A-75998. In an embodiment, the GnRH antagonist comprises abarelix. In an embodiment, the GnRH antagonist comprises degarelix. In an embodiment, the GnRH antagonist comprises relugolix. In an embodiment, the GnRH antagonist is Cetrorelix.

As used herein, “Cetrorelix” (trade name: Cetrotide™) refers to the synthetic decapeptide acetyl-D-3-(2′-naphtyl)-alanine-D-4-chlorophenylalanine-D-3-(3′-pyridyl)-alanine-L-serine-L-tyrosine-D-citruline-L-leucine-L-arginine-L-proline-D-alanine-amide (SEQ ID NO: 2); IUPAC name: N-acetyl-3-(2-naphthyl)-D-alanyl-4-chloro-D-phenylalanyl-3-(3-pyridyl)-D-alanyl-L-seryl-L-tyrosyl-D-citrullyl-L-leucyl-L-arginyl-L-prolyl-D-alaninamide.

As used herein′ “abarelix” (Plenaxis™) refers to N-acetyl-3-(2-naphthyl)-D-alanyl-4-chloro-D-phenylalanyl-3-(3-pyridyl)-D-alanyl-L-seryl-N-methyl-L-tyrosyl-D-asparagyl-L-leucyl-N6-isopropyl-L-lysyl-L-prolyl-D-alaninamide.

As used herein “degarelix” (trade name: Firmagon™) refers to N-acetyl-3-(2-naphthyl)-D-alanyl-4-chloro-D-phenylalanyl-3-(3-pyridyl)-D-alanyl-L-seryl-4-((S)-dihydroorotamido)-L-phenylalanyl-4-ureido-D-phenylalanyl-L-leucyl-N6-isopropyl-L-lysyl-L-prolyl-D-alaninamide.

As used herein “relugolix” (trade names: Orgovyx™, Relumina™) refers to 1-[4-[1-[(2,6-difluorophenyl)methyl]-5-[(dimethylamino)methyl]-3-(6-methoxypyridazin-3-yl)-2,4-dioxothieno[2,3-d]pyrimidin-6-yl]phenyl]-3-methoxyurea.

The term “follicular wave” refers to a phase in the ovarian cycle which corresponds to the simultaneous growth of multiple ovarian follicles, followed by the selection and continued growth of one or more dominant follicles (depending on the species) and regression of the remaining, subordinate follicles. Accordingly, the terms “follicular wave emergence”, “FWE”, “wave emergence” and variants thereof refer to the beginning or early stages of a follicular wave. In cattle and alpacas, FWE can be defined retrospectively as the first detection of the dominant follicle at 4 or 5 mm size with concomitant increase in number of 3-4 mm follicles.

The phrase “synchronizing follicular wave emergence”, “synchronize follicular wave emergence”, and variants thereof, when used with respect to a population of female mammals at differing (i.e. random) phases of ovarian cycle, means that follicular wave emergence occurs in individual animals at about the same time, or within the same time span, as the remaining animals in the population. For example, FWE may occur in an individual animal on the same day as, or within one or two days of, other animals in the population. As will be understood by the skilled person, synchronous FWE in a population of animals permits ovulatory induction methods to be synchronized in the population, thereby providing synchronous ovulation in the population of animals.

The time of FWE after administration of the GnRH antagonist will vary depending on various factors, such as the given drug or compound, the dose administered, the pharmaceutical formulation, the route of administration, the species of mammal being treated, and the like. The timing of FWE for a given combination of factors (e.g. species, drug, dose, etc.) can be determined experimentally using methods known in the art, for example using ultrasound (e.g. repeated ultrasound examinations at 12 to 24 hour intervals).

As used herein, the term “fixed time” means a specific or predetermined amount of time between one event, such as a stage or a step of a method, and another event, such as another stage or another step in the method. As will be understood by the skilled person, the timing of subsequent events or steps (“fixed time”) in the methods described herein will depend on various factors as indicated above (e.g. species, drug, dose, etc.). By way of example, new FWE occurs at a fixed time following the administration of a GnRH antagonist. In the Examples shown herein, in heifers, new FWE is observed at a fixed time of about 3.40±0.75 days after administration of a single intramuscular dose of 20 ug/kg body weight of Cetrorelix acetate, about 3.5±0.4 days after administration of a single intramuscular dose of 3 mg (about 7.5 ug/kg body weight) Cetrorelix, or about 5.3±0.3 days after administration of the first dose of two 1.5 mg (about 3.75 ug/kg body weight) intramuscular doses of Cetrorelix. In the Examples shown herein, in alpacas, new FWE is observed at a fixed time of about 7.92±0.24 days after administration of the first dose of two 1.5 mg intramuscular doses of Cetrorelix acetate or about 5.33±0.70 days after a single intramuscular injection of 1.5 mg cetrorelix.

For methods described herein involving superstimulatory or superovulatory methods, “administering an effective amount of FSH or an FSH agonist to the female mammal at a fixed time following the administration of the GnRH antagonist” means that FSH or an FSH analog is administered starting at about the expected time of FWE (resulting from the administration of the GnRH antagonist) and then continued for 3 to 7 days depending on the mammalian species. In an embodiment, FSH may be given by intramuscular injections at 12 hr intervals for 4 days or 7 days starting at the time of FWE. As will be understood, FSH may block regression of an extant dominant follicle. Accordingly, for the purpose of inducing new wave emergence and/or synchronizing follicular wave emergence for initiating superstimulation or superovulation, FSH should not be co-administered with the GnRH antagonist. In the methods described herein, FSH should only be administered after endogenous LH and/or FSH levels are reduced to baseline levels, and/or any extant dominant follicles are expected to have regressed and a new follicular wave has been induced following administration of the GnRH antagonist.

As will be understood in the art, in some cases, for example in species that are spontaneous ovulators (e.g. bovine, ovine, equine, porcine, canine), individual mammals in the population may have an existing corpus luteum which could interfere with synchronization. Accordingly, to circumvent the need to examine individual animals for the presence of an existing functional corpus luteum, additional treatments may be administered. For example, progesterone may be co-administered with the GnRH antagonist a) to prevent LH surge and ovulation, b) to prime uterine endometrium to prevent early PGF release after ovulation, and c) progesterone withdrawal sets up ovulatory cascade. Similarly, PGF may be administered about the time of progesterone withdrawal to induce luteolysis of any existing corpus luteum, which further leads to decrease in plasma progesterone levels and triggers ovulatory cascade if a growing or early static phase dominant follicle is present at that time. Accordingly, for the methods described herein, “administering prostaglandin (PGF) or a prostaglandin analog at a fixed time following the administration of the GnRH antagonist” or “administering prostaglandin (PGF) or a prostaglandin analog at a fixed time following the administration of the FSH” means administering PGF or a PGF analog about the time that a functional corpus luteum would be susceptible to PGF-induced luteolysis. Similarly, the administration of progesterone or a progesterone analog would be withdrawn at about the time that the initiation of an ovulatory cascade is desired (e.g. when a growing or early static phase dominant follicle is expected to be present following FWE resulting from administration of the GnRH antagonist). For example, in the case of heifers administered a single intramuscular dose of 20 ug/kg body weight of Cetrorelix acetate, PGF or a PGF analog is administered at a fixed time of about 7-12 days, or optionally about 7-8 days after administration of the GnRH antagonist (or about 3-7 days, or optionally about 3-4 days after FWE). Similarly, the progesterone or progesterone analog is withdrawn about 7-12 days or optionally about 7-8 days after administration of the GnRH antagonist (or about 3-7 days, or optionally about 3-4 days after FWE) in cattle. Optionally, the progesterone or progesterone analog is withdrawn about 0-48 hours after administration of the PGF or PGF analog. Suitable times for administering the PGF or PGF analog, and/or withdrawing the progesterone or progesterone analog can be determined by the skilled person.

As will be understood in the art, an ovulation inducing agent is preferably administered when a preovulatory dominant follicle is present. Accordingly, for the methods described herein, “administering an effective amount of an ovulation inducing agent at a fixed time following the administration of the GnRH antagonist” or “administering an effective amount of an ovulation inducing agent at a fixed time following the administration of FSH” means administering the ovulation inducing agent at about the time that a preovulatory dominant follicle has developed following FWE resulting from the administration of the GnRH antagonist. For example, in cattle a preovulatory dominant follicle is expected to develop about 9-14 days after administration of the GnRH antagonist (or about 5-7 days after expected FWE), or about 5-9 days after administration of FSH. Accordingly, the ovulation inducing agent may be administered about 9-14 days after administration of the GnRH antagonist (or about 5-7 days after expected FWE), or about 5-9 days after administration of FSH. For the methods described herein, “administering an effective amount of an ovulation inducing agent at a fixed time following the administration of the PGF or PGF analog”, “administering an effective amount of an ovulation inducing agent at a fixed time following withdrawal of the progesterone or progesterone analog”, or “administering an effective amount of an ovulation inducing agent at a fixed time following the withdrawal of the progesterone or progesterone analog and/or administration of the PGF or PGF analog” means administering an ovulation inducing agent after luteolysis of any preexisting corpus luteum and/or preovulatory/LH surge, which follows the administration of the PGF or PGF analog and/or withdrawal of the progesterone or progesterone analog. For example, in the case of heifers, luteolysis of the corpus luteum and/or a preovulatory/LH surge occurs about two days after administration of the PGF or PGF analog and/or withdrawal of the progesterone or progesterone analog. Accordingly, the ovulation inducing agent may be administered about 2 days after the withdrawal of progesterone or progesterone analog and/or administration of the PGF or PGF analog. Suitable times for administering the ovulation inducing agent can be determined by the skilled person.

For methods described herein involving breeding/mating or artificial insemination, a “fixed time following the administration of the ovulation inducing agent” means the mating or artificial insemination is carried out within a period of time relative to expected ovulation resulting from administration of the ovulation inducing agent, for example before expected ovulation, about the time of expected ovulation, and/or within a period of time after expected ovulation. The timing of ovulation may depend on the ovulation inducing agent, dose, and species of animal, and the timing of mating or artificial insemination (before, about, and/or after the time of ovulation) depends on the species. For example, in alpacas ovulation occurs about 24 to 30 hr after administering LH (irrespective of mating), and mating or artificial insemination of alpacas is carried out about 12-24 hours before ovulation. Accordingly, mating or artificial insemination of alpacas may be carried out 0 to 18 hours following the administration of LH, for example about 12 hours after the administration of LH. Mating or artificial insemination may be carried out about 12-24 hr before ovulation in, for example, cattle, sheep, goats, and alpacas, or about 24-36 hr after ovulation in, for example, dogs. In cattle, mating or artificial insemination is carried out up to about one day following the administration of the ovulation inducing agent, for example about the same time as, about 12 hours following, and/or about 24 hours following the administration of the ovulation inducing agent (e.g. GnRH or LH). As will be understood, suitable times for mating or artificial insemination will depend on the species of mammal, and can nevertheless be determined by the skilled person.

For methods described herein involving oocyte collection, “collecting one or more oocytes at a fixed time following the administration of the GnRH antagonist”, or “collecting one or more oocytes at a fixed time following the administration of the FSH or FSH agonist”, or “collecting one or more oocytes at a fixed time following the administration of the ovulation inducing agent” means that oocytes are collected at the time they are expected to be the desired stage of development. For example immature oocytes may be collected about 2-4 days after expected FWE if FSH is not used, or about 3-9 days after FWE if FSH is administered. Oocytes may be collected using any suitable method, for example oocytes (including cumulus oocyte complexes) and follicular fluid may be aspirated using vacuum from the ovaries by ultrasound-guided puncturing with a needle. For example, in cattle, oocytes may be collected 6-11 days after GnRH antagonist treatment without giving FSH treatment, or 3-9 days after start of FSH treatment, or 30-48 hours after withdrawal of progesterone or administration of PGF, or 0 to 24 hr after giving intramuscular injection of ovulation inducing agent such as GnRH or LH. As will be understood, suitable times for oocyte collection depend on the species of mammal and desired stage of oocyte development, and can nevertheless be determined by the skilled person.

For methods described herein involving embryo collection, “collecting one or more embryos at a fixed time following artificial insemination” or “collecting one or more embryos at a fixed time following mating” means that embryos are collected when they are expected to be at a morula or blastocyst stage. Embryos may be collected from the uterus using any suitable method, for example a surgical method or non-surgically by placing a catheter into the uterus or uterine horn through vagina and cervix. Embryos may be collected for example about 5 to 10 days after artificial insemination. For example, in cattle, embryos are collected 7, 8 and/or 9 days after artificial insemination. As will be understood, suitable times for embryo collection depend on the species of mammal and desired stage of embryo development, and can nevertheless be determined by the skilled person.

For methods described herein involving embryo transfer, “transferring an embryo to the female mammal at a fixed time following the administration of the ovulation inducing agent” means that an embryo is transferred after ovulation and subsequent development of a corpus luteum that produces progesterone, which may be for example about 2 to 8 days after the expected time of ovulation, or about 3 to 10 days after administration of the ovulation inducing agent, depending on the species. Any suitable embryo may be transferred, including a fresh/unfrozen or frozen-thawed in-vivo collected embryo or an in-vitro produced embryo. Embryos may be transferred using any suitable methods known in the art, for example the embryo may be transferred into the uterus or uterine horn by surgical method or non-surgically by placing a catheter into the uterus or uterine horn through vagina and cervix. Embryos may be transferred to the recipient for example about 3 to 10 days after administering the ovulation inducing agent, depending on the species and stage of embryo. For example, in cattle, embryos may be transferred 7, 8 or 9 days after administering LH, GnRH or estradiol. As will be understood in the art, progesterone (e.g. produced by the corpus luteum) acts on the uterine endometrium which develops in synchrony with any developing embryos, and accordingly the age of the embryo should match (within e.g. about 24 hours) the number of days after ovulation. For example, if an embryo is transferred to a recipient about 7 days after ovulation, the embryo should be at about 7 days of development. For example, in humans, zygotes can be transferred directly into the uterus 2 days after ovulation, and blastocysts can be transferred to the uterus 5 to 6 days after ovulation. Accordingly, it should be understood that suitable times for embryo transfer depend on a number of factors including the species of mammal, the ovulation inducing agent, and the stage of embryo being transferred, and can nevertheless be determined by the skilled person.

As used herein, the term “mammal” is used suitably to refer to animal species in which female animals undergo ovulatory cycles (e.g. estrus). Unless the context clearly dictates otherwise, the term “animal” is also used herein to refer to mammalian animals, and in some contexts is used to refer particularly to female mammals, including humans. The term “domestic animal” refers to species of mammal commonly bred or kept in captivity, such as for example livestock/beasts of burden (e.g. bovines such as cattle and water-buffaloes, camelids such as llamas, alpacas and camels, ovine (e.g. sheep), caprine (e.g. goats), porcine (e.g. pigs), equine (e.g. horses and donkeys)), and pets (e.g. cats, dogs), or in particular, female animals of these species. Similarly, the term “wildlife animal” encompasses semi-domesticated and non-domesticated mammal species such as, for example, bison, elk, caribou, deer (e.g. white-tail deer), muskox, non-human primates, canines, and felines such as tigers, lions, panthers or female animals of these species. In an embodiment, the mammal is a domestic animal, optionally a bovine, camelid, ovine, caprine, porcine, canine, or feline. In an embodiment, the mammal is a wildlife animal, optionally a bison, elk, caribou, deer, muskox, non-human primate, canine, or feline. In an embodiment, the mammal is a human.

As used herein, the term “mixed population” means a population of female mammals that are asynchronous with respect to their phase of ovulatory cycle.

As used herein, the term “oocyte” means a female reproductive cell of any stage which can give rise to an embryo once fertilized, and includes, without limitation, oocytes, ova, and cumulus-oocyte complexes (e.g. oocytes surrounded by compact or expanded layers of granulosa/cumulus cells).

As use herein, the term “embryo” means an animal in the early stages of development, for example a zygote, morula, or blastocyst.

The term “administered” or “administering” as used herein means administration of an effective amount of a compound or composition of the disclosure to an animal. The term “co-administration” or “combination therapy” shall mean that at least two compounds or compositions are administered to the animal such that effective amounts or concentrations of each of the two or more compounds may be found in the animal at a given point in time. Although compounds according to the present disclosure may be co-administered to an animal at the same time, the term embraces both administration of two or more agents at the same time or at different times, provided that effective concentrations of all co-administered compounds or compositions are found in the animal at a given time.

As used herein, the phrase “effective amount” means an amount effective, at dosages and for periods of time necessary, to achieve the desired result. For example in the context of follicular wave emergence (FWE), an effective amount is an amount that induces FWE in a female mammal at an approximate fixed time (or within a consistent time frame, for example within (±) one or two days) after administration, compared to the response obtained without administration of the compound. Effective amounts may vary according to factors such as the species, age, reproductive status, and/or weight of the animal. The amount of a given compound that will correspond to such an amount will vary depending upon various factors, such as the given drug or compound, the pharmaceutical formulation, the route of administration, the species of mammal being treated, and the like.

The GnRH antagonist may be administered according to any suitable schedule, for example as a single dose, or multiple doses (e.g. two doses), separated by a suitable period of time (e.g. about one day), or any other suitable number of doses and/or period of time, depending for example on the given drug or compound, formulation, route of administration, and/or species of mammal. In an embodiment, the GnRH antagonist is administered as a single dose. In an embodiment, the GnRH antagonist is administered as two doses about one day apart. In an embodiment, the GnRH antagonist is administered as a single dose in a dosage form that provides extended or continuous release over a period of time, for example over a period of about one day, about two days, about three days, or longer.

The GnRH antagonist can be administered by any suitable route of administration and using any suitable dosage form, for example, by parenteral, intravenous, subcutaneous, intramuscular, intraperitoneal, inhalation or spray (e.g. via aerosol), mucosal administration, rectal administration, vaginal administration, skin patch or skin application or oral administration, in dosage unit formulations containing conventional non-toxic pharmaceutically acceptable carriers, adjuvants and vehicles.

The composition comprising a GnRH antagonist such as Cetrorelix may be provided in any suitable dosage form. The term “dosage form” as used herein refers to the physical form of a dose for example comprising a compound of the disclosure, and includes without limitation injectable dosage forms, including, for example, sterile solutions and sterile powders for reconstitution, and the like, that are suitably formulated for injection; and liquid and solid dosage forms including, for example tablets, caplets, gelcaps, capsules, ingestible tablets, buccal tablets, troches, elixirs, suspensions, syrups, wafers, resuspendable powders, skin patch, intravaginal or subcutaneous implants, impregnated silicone devices, liquids and solutions. For example the injectable dosage form can be a subcutaneous, intradermal, or intramuscular depot injection that allows the compound to be released in a controlled and consistent way over a period of time, for example over several days or several weeks. Methods for making depot injections are described, for example, in U.S. Pat. No. 3,089,815 entitled “Injectable pharmaceutical preparation, and a method of making same” and herein incorporated by reference in its entirety. Dosage forms may also include those suitable for mucosal administration, including, for example, gels, creams, ointments, foams, tablets, or capsules, or via an insert comprising, for example, a substrate (e.g. silicone coating, tampon, or sponge) coated or impregnated with the GnRH antagonist, suitably for vaginal administration or skin patch.

Examples of suitable dosage ranges for Cetrorelix may include for example about 2 ug/kg body weight to about 200 ug/kg body weight, about 5 ug/kg body weight to about 100 ug/kg body weight, about 7.5 ug/kg body weight to about 75 ug/kg body weight, about 20 ug/kg body weight to about 50 ug/kg body weight, or any suitable dosage or dosage range between about 2 ug/kg body weight to about 200 ug/kg body weight, such as about 2.5 ug/kg body weight, about 3 ug/kg body weight, about 4 ug/kg body weight, about 5 ug/kg body weight, about 7.5 ug/kg body weight, about 10 ug/kg body weight, about 15 ug/kg body weight, about 20 ug/kg body weight, about 25 ug/kg body weight, about 30 ug/kg body weight, about 35 ug/kg body weight, or about 40 ug/kg body weight when administered by intramuscular injection. In an embodiment, the effective dose of Cetrorelix is about 7.5 ug/kg body weight administered by intramuscular injection. In an embodiment, the effective dose is about 40 ug/kg body weight administered by intramuscular injection. The dose may be administered as a single dose, or in multiple doses separated by a period of time such as for example a 24 hour period. In an embodiment, about 7.5 ug/kg body weight of Cetrorelix is administered as a single intramuscular dose. In an embodiment, about 7.5 ug/kg body weight of Cetrorelix is administered as two intramuscular doses of about 3.75 ug/kg body weight given about 24 hours apart. In an embodiment, about 20 ug/kg body weight of Cetrorelix is administered as a single intramuscular dose. In an embodiment, about 40 ug/kg body weight of Cetrorelix is administered as two intramuscular doses of about 20 ug/kg body weight given about 24 hours apart.

The GnRH antagonist may be administered or used in combination with one or more additional drugs to provide enhanced or additional reproductive management control, for example for inducing ovulation at a fixed time following administration of the GnRH antagonist (which can be done synchronously in a population of mammals thereby synchronizing ovulation). Controlled or synchronous ovulation has applications in breeding management, fixed-time artificial insemination protocols, or embryo transfer protocols. For example, the GnRH antagonist may be used, optionally in combination with progesterone or a progesterone analog, along with subsequent administration of prostaglandin (PGF) or a prostaglandin analog, and/or subsequent administration of an ovulation inducing agent, to delay and/or enhance synchronization of ovulation subsequent to the GnRH antagonist-induced follicular wave. Such combinations may be particularly suited for fixed-time artificial insemination or embryo transfer protocols. Accordingly, in an embodiment, the method further comprises administering an effective amount of one or more additional drugs. In an embodiment, the one or more additional drugs comprises progesterone or a progesterone analog, prostaglandin (PGF) or a PGF analog, and/or an ovulation inducing agent. Other drugs that may be used in addition to the GnRH antagonist in the methods described herein include, without limitation, LH, GnRH, PGF2a, FSH, equine choriogonadotropin (eCG), human choriogonadotropin (hCG), and estradiol benzoate, depending on the intended use. For example, fixed-time AI and embryo transfer differ in use of FSH, dose of PFG2a and LH/GnRH. For oocyte collection, LH can be omitted.

Progesterone and progesterone analogs may be administered using a progesterone drug releasing device, including commercial products such as a controlled internal drug release (CIDR) device (Zoetis), a PRID™ (CEVA), Cue-Mate™ (Vetoquinol) and subcutaneous devices such as syncromate-B. Progesterone analogs such as Norgestomet or oral supplementation with MGA (Melengestrol Acetate) may also be used in combination with the GnRH antagonist.

Prostaglandin (PGF) and PGF analogs include, without limitation, prostaglandin 2 alpha (PGF2a), cloprostenol, dinoprost, dinoprost tromethamine, bimatoprost, travoprost, carboprost and latanoprost.

As used herein, “ovulation inducing agent” means an agent that when administered to a female mammal, leads to ovulation of any extant preovulatory dominant follicle(s) present in the female mammal. Ovulation inducing agents include, without limitation, GnRH and GnRH analogs and agonists (e.g. buserelin, deslorelin, fertirelin, gonadorelin, goserelin, leuprorelin, triptorelin), LH and LH analogs, agonists, conjugates, and recombinant products (e.g. human chroiogondotropin/hCG, equine chriogonadotropin/eCG), and estradiol and estradiol esters, analogs and agonists (e.g. estradiol-17beta, estradiol benzoate, estradiol valerate, estradiol cypionate). Suitable ovulation inducing agents may be selected by the skilled person, and may depend on the species of mammal. For example, suitable ovulation inducing agents for camelids include GnRH or LH, but not estradiol.

### III. Devices, and Kits

An aspect includes a drug releasing device comprising a GnRH antagonist. Suitably, the drug releasing device is configured to provide controlled release of an effective amount of the GnRH antagonist over a period of time, such as, for example, about one day to about three days. In an embodiment, the GnRH antagonist is Cetrorelix. In an embodiment, the drug releasing device further comprises progesterone, and optionally is configured to provide controlled release of an effective amount of progesterone over an extended period of time, such as, for example, at least about 5 days to at least about 10 days or longer, optionally, about 5 days, about 6 days, about 7 days, about 8 days, about 9 days, about 10 days, or longer.

An aspect includes a kit comprising a GnRH antagonist along with suitable container or packaging and/or instructions for the use thereof, such as, for example, for use in synchronizing FWE, and optionally ovulation, in a population of female mammals, for use in a fixed-time protocols, for example a fixed-time artificial insemination protocol, a fixed-time oocyte collection protocol, or a fixed-time embryo production or embryo transfer protocol, or for use in a gonadotropin-stimulation and superovulation treatment protocol, optionally for embryo production/transfer. In an embodiment, the GnRH antagonist is provided in a drug releasing device. In an embodiment, the kit further comprises an applicator or other suitable delivery device. In an embodiment, the kit further comprises one or more additional drugs, such as, for example, progesterone, PGF2A or its analogs such as cloprostenol, GnRH, eCG and/or LH.

### III. Examples

The following non-limiting examples are illustrative of the present application:

### Example 1. Effect of GnRH Antagonist (Cetrorelix) on Response to Kisspeptin Treatment

Kisspeptin is a neuropeptide product of kiss-1 gene that is cleaved and/or degraded in 54-, 14-, 13- and 10-amino acid peptides (Kotani et al. 2001). GnRH and kisspeptin immunoreactive cells are located in close association in the preoptic area and arcuate nucleus of the hypothalamus in mice, primates, sheep (Ramaswamy et al. 2011; Smith et al. 2008; Clarkson & Herbison, 2006) and cattle (Tanco et al. 2016). Peripheral injections of kisspeptin induce luteinizing hormone (LH) secretion and ovulation in several mammalian species (Caraty et al. 2007; Caraty et al. 2013; d'Anglemont de Tassigny et al. 2008; Ezzat Ahmed et al, 2009; Matsui et al. 2004) including cows (Leonardi et al. 2019; Leonardi et al. 2018) and seasonally anestrus sheep Caraty et al. 2007). Further, intravenous administration of kisspeptin-10 induces the release of GnRH into the hypophyseal portal circulation in sheep (Smith et al. 2011). It is unknown if the 10-amino acid kisspeptin fragment (kisspeptin-10) is able to cross the blood-brain barrier to stimulate GnRH neurons after peripheral injection in cattle.

It is not yet known if peripheral kisspeptin can activate the GnRH neurons in cattle.

Endogenous and exogenous GnRH induces release and synthesis of LH from the pituitary gland in cattle (Vizcarra et al. 1997; Yoshioka et al. 2001) and administration of a GnRH antagonist prevents the LH surge (Ginther et al. 2012). Peripheral administration of kisspeptin increases plasma LH concentration in ovariectomized cows (Whitlock et al. 2008) and pubertal heifers (Kadokawa et al. 2008), and induces ovulation under a low-progesterone milieu (Leonardi et al. 2019). In a previous study, a progressive increase in LH secretion was detected within 15 minutes after repeated intravenous administration of kisspeptin-10 in cows with plasma progesterone below 1.7 ng/ml (Leonardi et al. 2019). However, the mechanism by which peripheral administration of kisspeptin-10 induces LH release in cows is unclear. In vitro studies in horses and cattle raise the possibility of direct LH release from anterior pituitary cells by kisspeptin (Ezzat et al. 2010; Magee et al. 2020). Whether kisspeptin-10 crosses the blood-brain barrier to stimulate GnRH neuronal cell bodies, or acts on GnRH nerve terminals in median eminence, or the observed LH release is due to a direct effect on the pituitary gonadotrophs in vivo remains unknown.

Objective: To determine if pre-treatment with a GnRH antagonist would alter the pattern of kisspeptin-induced LH release and ovulation. Cetrorelix was used as a blocking agent for the action of Kisspeptin in a low plasma progesterone persistent dominant follicle model.

Hypothesis: 1) Pre-treatment with a GnRH antagonist (Cetrorelix) before kisspeptin treatment would suppress LH release and prevent ovulation in cattle.

Methods: The human Kisspeptin-10 (hKP-10) peptide (YNWNSFGLRF-NH2; SEQ ID NO: 1) was custom synthetized at >95% of purity (MW: 1318.44 g/mol) by GenScript USA Inc, Piscataway, NJ, USA. The sequence is based on the predicted C-terminal region (112-121-NH2) of human metastin (Gen Bank accession #AY117143) and has been previously used in cattle. The peptide was previously tested for solubility and was dissolved in ultrapure water at 10 mg/mL.

The study was performed on Hereford crossbred heifers (n=15; 500±24 Kg body weight, 17-18 month age) that had a corpus luteum in one of the ovaries at the start of experiment. The heifers were maintained in outdoor pens at the University of Saskatchewan Goodale Research Farm (52° north and 106° west). Heifers were fed barley silage and had hay and water ad libitum. A mineral salt block was available to heifers throughout the study period. All procedures were performed in accordance with Canadian Council on Animal Care and were approved by the University of Saskatchewan Protocol Review Committee.

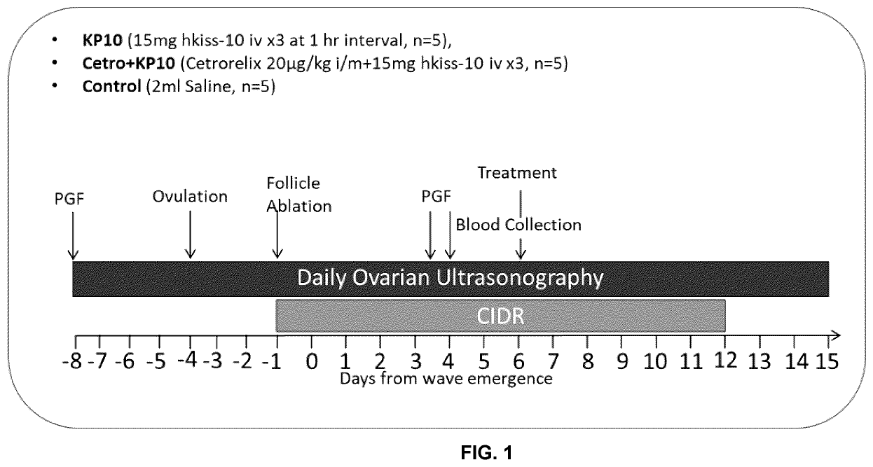

A schematic diagram of the experimental design is shown in FIG. 1. Heifers (n=15) were selected from a larger group based on the detection of a corpus luteum (CL) by transrectal ultrasonography. Immediately after ultrasound examination, heifers were given PGF2a twice at an interval of 12-hours. The low-progesterone bovine model described previously (Leonardi et al. 2019) was used to test the hypothesis. Briefly, the ovaries were examined daily by ultrasonography to detect ovulation. Three days after ovulation, follicles ≥5 mm in diameter in both ovaries were ablated by transvaginal ultrasound-guided follicle aspiration, inducing the emergence of new follicular wave 1.5 days after the procedure (Bergfelt et al. 1994). After follicular ablation, a progesterone device was placed in the vagina (CIDR; Zoetis). The day of wave emergence (Day 0) was defined as the day when the follicle of wave was first recorded (4 to 5 mm in diameter) with concurrent increase in diameter. Heifers were given PGF2α im on Days 3.5 and 4 (i.e., 8 and 8.5 days after ovulation) to cause luteolysis, however, the CIDR devices were left in place until Day 12 to maintain a low-progesterone milieu sufficient to prevent spontaneous ovulation. On Day 6, an indwelling jugular catheter was placed as described (Bergfelt et al. 1997) and heifers were assigned randomly to three groups (n=5 per group): 1) 3 iv doses of 15 mg human kisspeptin-10 at 1 h intervals (Kp10 group), 2) pretreatment with single im dose of Cetrorelix acetate (20 μg/Kg of body weight, GnRH antagonist—Sigma #C5249 diluted in 5% D-Manitol w/v, Sigma #M4125) followed 3 h later with 3 iv doses of 15 mg human kisspeptin-10 at 1 h intervals (Cetrorelix group), or 3) 3 iv doses of normal saline at 1 h intervals (Control group). Following treatment (on Day 6), ovarian ultrasound examinations were performed daily until the end of the experiment to detect ovulation of the extant dominant follicle (i.e., dominant follicle present at the time of treatment) or of the dominant follicle originating from a subsequent wave. The progesterone device was withdrawn on Day 12 (i.e., thirteen days after insertion) and ultrasound examinations were continued until ovulation was detected.

Blood samples and hormone assays: Serial blood samples on the day of treatment were collected at 15 minute intervals from −30 to 150 min (0 min=time of first hKP-10 or saline injection) using the indwelling jugular catheter. All blood samples were collected in heparinized tubes (Vacutainer, BD, Franklin Lakes NJ, USA). Immediately after sampling, tubes were centrifuged at 1,500×g for 15 min, and plasma was separated and stored at −20° C.

The plasma samples from both experiments were analyzed for LH and progesterone concentration at the University of Wisconsin (Madison, WI, USA) in Dr O. J. Ginther's Research Laboratory. Plasma LH concentrations were measured with a validated radioimmunoassay for cattle (Bolt et al. 1990) with modifications as reported Ginther et al. 1999). Briefly, LH concentrations were measured in duplicate using USDA-bLH-B-6 for ¹²⁵I-iodination and for preparing reference standards, and USDA-309-684P as the primary antibody (National Hormone and Pituitary Program, Torrance, CA, USA). The standard curve ranged from 0.078 to 20.0 ng/ml with sensitivity of 0.1 ng mL⁻¹. Intra- and inter-assay coefficients of variation and mean sensitivity were 6.23%, 12.24% and 0.03 ng/ml, respectively. Progesterone concentrations were measured as described (Ginther et al. 2005) in a single assay batch with a commercial solid-phase RIA kit containing antibody-coated tubes and ¹²⁵I-labeled progesterone (ImmuChem Coated Tube progesterone 125 RIA kit, MP Biomedical, Costa Mesa, CA). The intra-assay coefficients of variation and sensitivity for progesterone were 11.97% and 0.06 ng/ml, respectively.

Statistical Analyses: Data analyses were performed using SAS (Statistical Analysis System, software package 9.4, SAS Institute Inc., Cary, NC, USA). In both experiments, single-point measurements (i.e., diameter of the dominant follicle at the time of treatment and 24 hours after treatment; diameter of the CL at the time of treatment and progesterone concentration at the time of treatment) were analyzed using one-way analysis of variance. Statistical significance was assumed when the P-value was ≤0.05 whereas a tendency for a difference was assumed when the P-value was between >0.05 to ≤0.1. Tukey's post-hoc test was used for multiple comparisons if the P-value for a test detected a difference. Ovulation rate was analyzed using the GLIMMIX procedure.

Analyses of repeated measures data (e.g., LH plasma levels, follicular dynamic) were performed using MIXED models procedure, in which treatment, time, and treatment-by-time interaction were tested and a repeat statement was included in the syntax (repeated days subject=cowID). Initial analyses tested five covariance structures (SIMPLE, CS, AR (1), ANTE(1), or UN) and the model with smallest AICC value was selected for final analysis. All values are reported as mean±SEM.

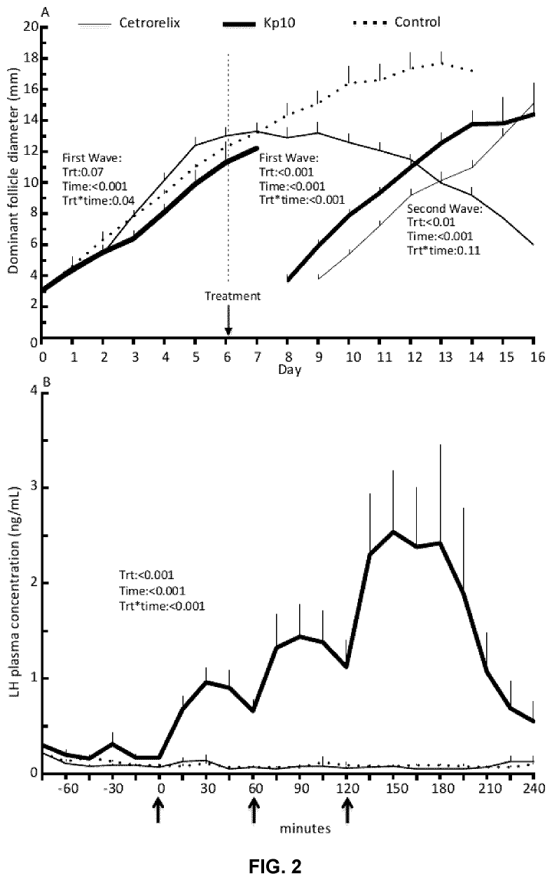

Results: The diameter of the dominant ovarian follicle at the time of treatment (Day 6 of follicular wave) and 24 h after treatment did not differ among the Kp10, Cetrorelix and Control groups (Table 1). Plasma progesterone concentrations were similar among groups at the time of treatment. The Kp10 group had higher plasma LH concentrations than the Cetrorelix and Control groups (P<0.001; FIG. 2B). Plasma LH concentrations remained higher in the Kp10 group than the other two groups from 15 to 240 min (0 min=first injection of the treatment; FIG. 2A). Ovulation of the extant dominant follicle was recorded in 4 of 5 heifers in the Kp10 group compared to 0 of 5 heifers in the Cetrorelix and Control groups (Table 1). A new follicular wave emerged immediately in all four heifers that ovulated after kisspeptin treatment (FIG. 2A). Regression of the extant dominant follicle was recorded 3 to 4 days after treatment in the Cetrorelix group (FIG. 2A). Emergence of a new follicular wave occurred 3.40±0.75 days after treatment in the Cetrorelix group (i.e., without ovulation of extant dominant follicle) compared to 2.75±0.75 days in the Kp10 group (after ovulation of extant dominant follicle in 4 of 5 heifers). In the Control group, the extant dominant follicles did not regress, and ovulated on Day 15 after CIDR removal (withdrawn on Day 12). All new dominant follicles from the Kp10 (n=4) and Cetrorelix (n=5) group ovulated on Day 15 after the CIDR removal (Day 12).

TABLE 1
Ovarian and endocrine responses (mean ± SEM) of heifers treated
with human Kisspeptin-10 (Kp10 group), pre-treatment with Cetrorelix
before Kisspeptin-10 (Cetrorelix group) or normal saline (Control
group) intravenously under subluteal levels of plasma progesterone.

| | | | | |
|---|---|---|---|---|
| | Kp10 | Cetrorelix | Control | |
| Endpoint | (n = 5) | (n = 5) | (n = 5) | P-value* |

Plasma progesterone on the day of treatment (Day 6)

| | | | |
|---|---|---|---|
| 1.86 ± 0.43 | 1.41 ± 0.56 | 1.86 ± 0.38 | 0.73 |

Ovulation rate after treatments:

| | | | | |
|---|---|---|---|---|
| number of heifer/total | 4/5^{a} | 0/5^{b} | 0/5^{b} | 0.02 |
| hours after treatment^{¶} | 48 | — | — | |

Dominant follicle diameter (mm):

| | | | | |
|---|---|---|---|---|
| at time of treatment | 11.34 ± 0.91 | 13.01 ± 0.68 | 12.36 ± 0.26 | 0.26 |
| 24 h after treatment | 12.24 ± 0.89 | 13.38 ± 0.55 | 13.22 ± 0.34 | 0.42 |
| 48 h after treatment* | 16.51 | 12.91 ± 0.55 | 14.67 ± 0.56 | 0.06 |

Number of ovulations after CIDR removal on Day 12:

| | | | | |
|---|---|---|---|---|
| Extant dominant follicle*^{#} | 1/1 | 0/5 | 5/5 | 0.01 |
| New dominant follicle*^{†} | 4/4 | 5/5 | 0/0 | 0.81 |

^{a, b}Within rows, values with no common superscript are different (P ≤ 0.05)
*Statistical analysis for two treatment groups
^{¶}Interval between treatment and ovulation
^{#}Dominant follicle present at the time of treatment
^{†}Dominant follicle of a new wave that emerged after treatment

In 5 of 5 heifers in the Cetrorelix+KP10 group, a new wave emergence occurred at 3.40±0.75 days after treatment during the mid-static dominance phase under low progesterone milieu.

Wave emergence resulted due to regression of the extant dominant follicle caused by low plasma LH levels. Dominant follicle of the new wave was capable of ovulating after CIDR removal.

In all saline-treated control group heifers (n=5) dominance of the extant follicle was maintained and no new wave emergence occurred. Extant dominant follicle was capable of ovulating when CIDR device was removed on Day 12.

In 4 of 5 heifers in the KP10 group, a new wave emergence occurred at 2.75±0.75 days after treatment due to ovulation of the extant dominant follicle (resulting from elevated levels of plasma LH). CL resulting from ovulation of the extant dominant follicle were short-lived.

Conclusions: An unexpected downstream effect of LH suppression in Cetrorelix-treated heifers was also observed on the dominant follicle-all heifers failed to ovulate (0 ovulations of 5) in this group compared to 4 out of 5 heifers ovulating by 48 hours after kisspeptin treatment. Interestingly, the dominant follicle of all animals entered the regression phase by 72 hours after kisspeptin and GnRH antagonist treatment.

In conclusion, pre-treatment with a GnRH antagonist, Cetrorelix, 3 hours before initiation of intravenous treatments with kisspeptin suppressed the kisspeptin-induced LH surge, prevented ovulations, and caused regression of the extant dominant follicle followed by emergence of a new follicular wave, on average, 3.4 days after treatment in all five heifers. The likely mechanism of kisspeptin-mediated LH release is downstream of GnRH synthesis, perhaps by inducing release of pre-synthesized GnRH from the nerve terminals in the median eminence.

### Example 2. Alpaca Cetrorelix Known Stage Study

Cetrorelix inhibits the preovulatory LH surge induced by ovulation inhibiting factor (OIF) in llamas, suggesting that LH secretion is modulated by a direct or indirect effect of OIF on GnRH neurons in the hypothalamus (Silva et al 2011). It is unclear whether the effects observed on the dominant follicle in Example 1 also applies to llamas.

Moreover, it is also unclear if the same effects could be observed during other stages of follicular development (growing, static, regressing preovulatory phases) and luteal development (metestrus, diestrus and proestrus).

Objective: Identify the pattern of growth for the dominant follicle in camelids following treatment with a GnRH antagonist during different stages of the follicular wave.

Hypotheses: 1) The GnRH antagonist will cause regression of both the early/mid-growing phase and mid-static phase dominant follicle; and 2) A new wave will emerge at a consistent time after the treatment irrespective of the follicular status of the dominant follicle.

Methods: The specific objective of this experiment was to identify the pattern of growth for the dominant follicle in alpacas following treatment with a GnRH antagonist during two contrasting stages of the follicular wave. Based on data from natural spontaneous waves in alpacas, the dominant follicle is expected to be in early- to mid-growing phase (6 to 7 mm in diameter) on Day 5 and in mid-static phase (9 to 10 mm) on Day 10, so the treatments were planned to begin on these days.

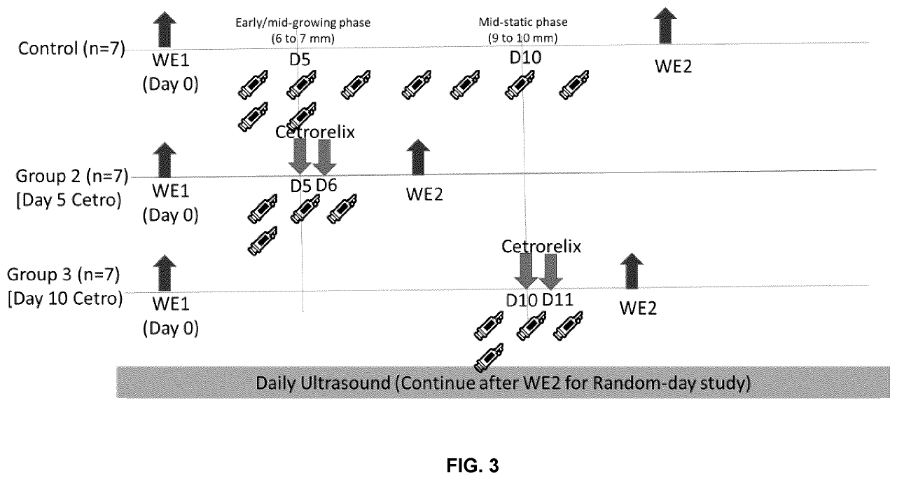

The experimental design is illustrated FIG. 3. Transrectal ovarian ultrasonography was performed using an Esoate My Lab 5 ultrasound machine and a 7.5 linear array transducer attached to a custom-build rigid PVC pipe handle. The pipe was slightly curved at the transducer end to facilitate manipulation of the transducer. The transducer and the plastic handle were lubricated using methyl cellulose ultrasound gel and inserted slowly into the rectum after manual removal of the faeces. Reproductive track and ovaries were located and 10 to 20 second video clips of left and right ovary were recorded. Sketches of ovaries were drawn daily at the time of imaging to record location, size and number of follicles >2 mm in diameter as well as the size and location of the corpus luteum (if present). Ultrasound examinations were performed daily. The experimental data recording began when a spontaneous emergence of a new follicular wave detected in an animal (defined as Extant follicular wave). The day of wave emergence was designated as Day 0 and was defined retrospectively as the first detection of the dominant follicle at 4 or 5 mm size with concomitant increase in number of 3-4 mm follicles.

Based on the day of wave emergence, alpacas were allocated to one of the three treatment groups (n=7 animals per group) using systematic random sampling method. The first group of alpacas (Control group) were given no treatment, their ovaries were examined daily and blood samples were obtained daily from Day 4 to 12 to compare hormonal data from analogous days after Cetrorelix treatment. The second group of alpacas (D5 group) were given the first intramuscular injection of 1.5 mg (3 mL of 0.5 mg per mL solution) Cetrorelix acetate on Day 5 and a second injection of 3 mL on Day 6 (i.e., two i/m injections of 1.5 mg Cetrorelix at 24 hour interval for a total dose of 3 mg). The Cetrorelix dose administered was approximately equal to 20 ug/Kg body weight per day (considering average body weight of alpaca=75 kg) and based on the previous studies in llama and cattle (Silva et al, 2011, Ulker et al 2001). Alpacas in the third group (D10 group) were given the first intramuscular injection of 1.5 mg Cetrorelix acetate on Day 10 and a second injection of 1.5 mg on Day 11.

Cetrorelix treatments were given during the extant follicular wave. Changes in the size of the dominant follicle, largest subordinate follicle, and the number of ≥3 mm follicles were recorded daily from spontaneous emergence of extant follicular wave to Day 6 of next (post-treatment) follicular wave emergence. The inter-wave interval was determined based on duration between emergence of successive follicular waves.

Blood samples (5 to 8 mL) were collected in heparinized tubes (BD Vacutainer, BD Franklin Lakes, NJ, USA) via jugular venipuncture. Blood samples were kept on ice packs till centrifugation at 200 rpm (within 1 hr of collection) to obtain plasma samples that were immediately frozen at −20° C. until measurement of plasma LH. Plasma samples were obtained on Day 4 to 7 in Group 2 (that is, 1 day before to 1 day after Cetrorelix treatment) and on Day 9 to 12 in Group 3. For direct comparison, plasma samples were obtained from Day 4 to 12 in control group.

Results: Results are presented in Table 2. Post-treatment data from one animal from the D10 group was excluded because ovulation occurred on Day 10 before treatment was initiated.

In the untreated control group (n=7), next wave emergence occurred on Day 14.57±0.81 after the first wave emergence, i.e., the inter-wave interval of the extant wave ranged from 13 to 19 days with a median of 14 days. On average, the dominant follicle entered the static phase on Day 7.6±0.55 (data combined from Control and D10 groups).

The mean day of onset of dominant follicle regression was earlier in the D5 group than in the D10 and Control groups (Day 8.7±0.89, Day 12.9±0.34, Day 13.6±0.90, respectively; P<0.001) and dominant diameter was smaller in the D5 group than in the D10 and Control groups (7.4±0.5 mm on Day 6.0±0.90, 10.9±0.70 mm on Day 10.3±0.68, 10.2±0.93 mm on Day 10.0±0.90; P=0.005).

The duration of the dominant follicle static phase was shortest in the D5 group, intermediate in D10 group and longest in the Control group (3.9±0.63 days, 4.9±0.40, 6.3±0.52; P=0.015).

The inter-wave interval was shorter in D5 group, intermediate in the Control group and longer in D10 group (12.7±0.36 days, 14.57±0.81, 17.71±0.52; P<0.001).

Combined among Group 2 and 3, wave emergence occurred on an average 7.92±0.24 days (range 7 to 9 days) after the first Cetrorelix injection.

Taken all data together, when Cetrorelix treatment was given on Day 5 of the wave (n=7), the dominant follicle stopped growing, had shorter static phase, attained maximum diameter earlier, and the onset of regression occurred earlier than the Control or D10 group. A new wave was induced 7.71±0.36 days later (on Day 12.7 of the extant wave; i.e., earlier than control group).

When Cetrorelix treatment was given on Day 10 of the wave, the dominant follicle regressed the same time as in the control, and a new wave started 8.17±0.31 days after Cetrorelix treatment (on Day 18.17 of the extant wave; i.e., delayed relative to the control group).

The synchrony of wave emergence (defined as the interval from first treatment to next wave emergence) was similar in D5 and D10 groups (7.7±0.36 days [range: 7-9 days] vs. 7.7±0.52 days [range: 7-9 day; P>0.99]).

TABLE 2
Follicular endpoints and wave emergence data (mean ± standard error of mean)
from untreated alpacas (control) and those treated twice with 1.5 mg Cetrorelix
acetate i/m at 24 hr interval starting on Day 5 (D 5) or Day 10 (D 10) of the
follicular wave. Extant follicular wave is defined as the wave during which
Cetrorelix treatment was initiated. Reported p-values (last column) were obtained
by GLM procedure in SAS. Different superscripts in a row indicate differences
(P < 0.05) among groups detected by Tukey's post-hoc comparisons.

| | | | | |
|---|---|---|---|---|
| | Control | D 5 | D 10 | |
| Groups | (n = 7) | (n = 7) | (n = 7) | P-value |

Extant Wave

| | | | | |
|---|---|---|---|---|
| Total Number of Follicles | 12.71 ± 1.71 | 10.71 ± 0.97 | 9.14 ± 1.39 | 0.219 |
| on Day of Emergence | | | | |

Dominant Follicle

| | | | | |
|---|---|---|---|---|
| Maximum Diameter (mm) | 10.21 ± 0.93^{a} | 7.36 ± 0.47^{b} | 10.93 ± 0.70^{a} | 0.006 |
| Maximum Diameter (days) | 10.00 ± 0.90^{a} | 6.00 ± 0.90^{b} | 10.29 ± 0.68^{a} | 0.003 |
| Diameter on Day of Cetrorelix | — | 6.64 ± 0.43^{a} | 10.36 ± 0.85^{b} | 0.002 |

Treatment (mm)

| | | | | |
|---|---|---|---|---|
| Diameter on Day 5 (mm) | 7.40 ± 0.44 | 6.64 ± 0.43 | 9.71 ± 1.77 | 0.141 |
| Diameter on Day 10 (mm) | 9.64 ± 0.91^{a} | 5.50 ± 0.87^{b} | 10.36 ± 0.85^{a} | 0.002 |
| End of Growth (days) | 7.29 ± 1.06^{a} | 4.86 ± 0.40^{b} | 8.00 ± 0.38^{a} | 0.012 |
| Onset of Regression (days) | 13.57 ± 0.90^{a} | 8.71 ± 0.89^{b} | 12.86 ± 0.34^{a} | <0.001 |
| Length of Static Phase (days) | 6.29 ± 0.52^{a} | 3.86 ± 0.63^{b} | 4.86 ± 0.40^{ab} | 0.015 |

Largest Subordinate Follicle

| | | | | |
|---|---|---|---|---|
| Maximum Diameter (mm) | 4.79 ± 0.32 | 4.57 ± 0.40 | 5.79 ± 1.02 | 0.401 |
| Maximum Diameter (days) | 3.57 ± 1.34 | 1.86 ± 1.01 | 6.00 ± 1.36 | 0.088 |
| Diameter on Day of Cetrorelix | — | 3.21 ± 0.21 | 4.00 ± 0.97 | 0.444 |

Treatment (mm)

| | | | | |
|---|---|---|---|---|
| Diameter on Day 5 (mm) | 3.21 ± 0.15 | 3.21 ± 0.21 | 4.21 ± 0.76 | 0.241 |
| Diameter on Day 10 (mm) | 3.36 ± 0.48 | 2.71 ± 0.81 | 4.00 ± 0.97 | 0.379 |

Interwave Interval

| | | | | |
|---|---|---|---|---|
| Interwave Interval (days) | 14.57 ± 0.81^{a} | 12.71 ± 0.36^{b} | 17.71 ± 0.52^{c#} | <0.001 |

Next Wave

| | | | | |
|---|---|---|---|---|
| Total Number of Follicles | 9.43 ± 1.00 | 13.14 ± 1.47 | 12.14 ± 1.08^{#} | 0.105 |
| on Day of Emergence | | | | |

Degree of Synchrony

| | | | | |
|---|---|---|---|---|
| Days from treatment to | — | 7.71 ± 0.36 | 7.71 ± 0.52^{#} | 1.000 |
| next wave emergence | | | | |

^{#}n = 6 alpacas

Conclusion: Dominant follicle regressed and emergence of the next wave was synchronized after 2 injections of Cetrorelix when treatments were initiated during the growing phase or the late static phase of the dominant follicle in alpacas.

### Example 3: Alpaca Random Day Study

Objective: To examine the effect of a single injection of Cetrorelix on dominant follicle regression and synchrony of next wave emergence in alpacas when treatment is initiated on a random day of the wave.

Hypothesis: A single treatment with GnRH antagonist will cause regression of dominant follicle at random stages of development and re-examined if a new wave will emerge at a consistent time after the treatment irrespective of the phase of dominance (i.e., similar to Example 2).

Methods: Ultrasonographic methods and follicular endpoint recording were similar to those described in Example 2. Follicular endpoints were obtained from alpacas (n=15) before and after treatment, therefore each animal acts as its own control. A fixed calendar day (July 16) was pre-assigned for Cetrorelix treatment. Daily transrectal ultrasonography was performed (n=15 alpacas) from May 31 to June 16. This examination period was used to record the variance in wave emergence from a fixed calendar date (May 31) without giving treatment (control period). Animals were at unknown/random day of the wave at the beginning of this 17-day time-window (i.e., longer than one natural inter-wave interval); therefore, all animals were expected to have at least one wave emergence encompassing this period. All alpacas were treated with a single intramuscular injection of 1.5 mg (3 mL) Cetrorelix acetate on July 16 and daily ultrasound examinations continued from the day of treatment until Day 3 after the next (post-treatment) wave emergence (treatment period). Synchrony of wave emergence was compared between the control period and the treatment period.

Statistical analyses: A Proc Mixed repeated measures analysis of variance (SAS version 9.4) was performed to compare serial data (follicle size and follicle numbers changes over days) and ANOVA or paired T-test were used for single-point numerical measurements. Count data were compared using Fisher's Exact test. Synchrony of the wave emergence was compared using Barlett's and Levene's tests of homogeneity of the variance.

Results: The results of Example 3 are presented in Tables 3-6.

TABLE 3
Days of wave emergence after no treatment (control period) or after
single i/m injection of 1.5 mg Cetrorelix (treatment period).

Dates

| | |
|---|---|
| May 1 to Jun. 16, 2021 | Jul. 16 to Aug. 1, 2021 |

Duration from Treatment to Wave Emergence (days)

| Alpaca ID | Untreated | Cetrorelix |
|---|---|---|
| A10 | 11 | 4 |
| A18 | 10 | 11 |
| A5 | 9 | 3 |
| A6 | 4 | 4 |
| A9 | 11 | 9 |
| A12 | 2 | 1 |
| A17 | 12 | 4 |
| A21 | 7 | 3 |
| Anew | 2 | 4 |
| A3 | 7 | 4 |
| A8 | 15 | 4 |
| A13 | 16 | 6 |
| A14 | 4 | 8 |
| A16 | 16 | 8 |
| A22 | 8 | 7 |

The emergence of the next wave occurred on an average 5.33±0.70 days after a single treatment with 1.5 mg Cetrorelix on a random day of the wave compared to 8.93±1.21 days without treatment (in control period).

TABLE 4
Statistical analysis of next FWE following Cetrorelix treatment and untreated controls.
Analysis Variable: duration

| | N | | Std | | | | Lower 95% | Upper 95% |
|---|---|---|---|---|---|---|---|---|
| Group | Obs | Mean | Error | Variance | Range | N | CL for Mean | CL for Mean |
| Cetro | 15 | 5.33 | 0.701 | 7.381 | 10 | 15 | 3.829 | 6.838 |
| Control | 15 | 8.93 | 1.213 | 22.067 | 14 | 15 | 6.332 | 11.535 |

TABLE 5
Bartlett's test for homogeneity of duration variance.

| Source | DF | Chi-Square | Pr > ChiSq |
|---|---|---|---|
| Group | 1 | 3.8654 | 0.0493 |

TABLE 6
Levene's test for homogeneity of duration variance
ANOVA of squared deviations from group means.

| Source | DF | Sum of Squares | Mean Square | F Value | Pr > F |
|---|---|---|---|---|---|
| Group | 1 | 1409.0 | 1409.0 | 5.65 | 0.0245 |
| Error | 28 | 6980.6 | 249.3 | | |

A lower variation in duration after Cetrorelix treatment (variance 7.38) than control period (22.067) indicates synchrony of wave emergence [Barlett test of homogeneity of variance p=0.049, Levene test p=0.025].

The confidence interval range was 3.8 to 6.8 days after Cetrorelix treatment compared to control CI of 6.3 to 11.5 days.

### Example 4: Effect of Cetrorelix on the Fate of Pre-Selection, Growing Versus Regressing Dominant Follicle in Beef Heifers

Specific Objective: The primary objective was to determine the growth pattern of the dominant and first subordinate follicle in cattle following treatment with a GnRH antagonist, Cetrorelix, during the pre-selection period (Day 1-2), growing phase (Day 3-4) and late-static/early regressing stage (Day 6-7) of the follicular wave. The secondary objective was to record changes in CL function and progesterone production.

Hypothesis: Treatment with a GnRH antagonist results in the loss of follicular dominance and the emergence of a new follicular wave at a consistent and predictable interval after treatment, regardless of the follicular stage at the time of treatment (i.e., pre-selection, growing, the late-static/early regressing phase of the dominant follicle).

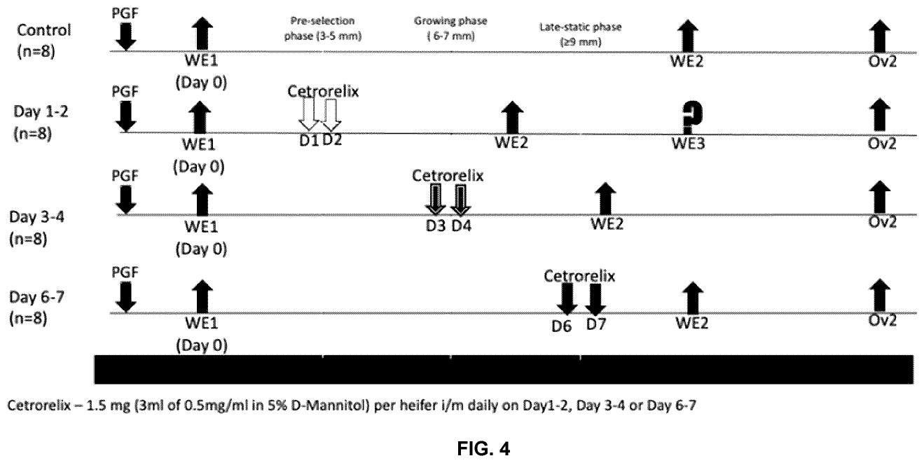

Experimental design: A schematic diagram of the experimental design is shown in FIG. 4. The study was conducted on pubertal Hereford-cross beef heifers (n=32) at the U. Sask. Livestock and Forage Centre of Excellence (LFCE). Initial transrectal ultrasonography was performed to confirm the puberty by the presence or absence of a corpus luteum (CL). Animals were also weighed at this time. Animals with a functional CL were administered an intramuscular (im) injection of prostaglandin F2a analog (cloprostenol) to cause CL regression and ovulation. Daily ultrasonography was performed to detect ovulation and to record the number, size and location of follicles and CL in both ovaries. The day of emergence of a follicular wave was initially estimated by sudden increase in the number of 3-4 mm follicles and confirmed retrospectively as the day on which the dominant follicle was first detected at 4 to 5 mm in size (defined as Day 0). Follicular and hormonal data (see next section for endpoints) from all animals was collected for one complete inter-ovulatory interval.

TABLE 7
Treatment Protocol (all treatments are given intramuscularly).

| Treatment Group | Day of the follicular wave (Day 0 = wave emergence) | | | | | |
|---|---|---|---|---|---|---|
| (Follicular phase) | Day 1 | Day 2 | Day 3 | Day 4 | Day 6 | Day 7 |
| Group1 (n = 8 heifers) | 3 ml saline | 3 ml saline | 3 ml saline | 3 ml saline | 3 ml saline | 3 ml saline |
| (Control group) | | | | | | |
| Group 2 (n = 8 heifers) | 1.5 mg | 1.5 mg | | | | |
| (Pre-selection Phase) | cetrorelix | cetrorelix | | | | |
| Group 3 (n = 8 heifers) | | | 1.5 mg | 1.5 mg | | |
| (Growing Phase) | | | cetrorelix | cetrorelix | | |
| Group 4 (n = 8 heifers) | | | | | 1.5 mg | 1.5 mg |
| (Late-static phase) | | | | | cetrorelix | cetrorelix |

*Cetrorelix intramuscular injection will be given in the neck as 3 ml of 0.5 mg/ml cetrorelix acetate in 5% D-Mannitol aqueous solution.

Animals were randomized into 4 groups based on the day of ovulation. Group 1 (control group) heifers were given 3 mL normal saline (sham-treated negative control; n=3 on Days 1 and 2, n=3 on Days 3 and 4, n=2 on Days 6 and 7). Group 2 heifers were given 1.5 mg Cetrorelix on Days 1 and 2 (pre-selection dominant follicle phase; n=8), Group 3 heifers were given Cetrorelix on Days 3 and 4 (dominant follicle growing phase; n=8), and Group 4 heifers were given Cetrorelix on Days 6 and 7 (dominant follicle late-static/early regressing phase; n=8). The dose of Cetrorelix (1.5 mg per treatment), route of administration (intramuscular) and duration of treatment (i.e., 2 doses at 24 hr interval) were selected based on Example 2 and previous studies in cattle (Ulker et al, 2001).

Changes in the size of the dominant follicle, largest subordinate follicle and CL, and the number of follicles in 3-5, 6-8, and 9+mm size categories were recorded daily from initial ovulation to the first post-treatment ovulation. The inter-wave interval was determined based on duration between emergence of successive follicular waves.

Blood samples were also collected every 12 hours by jugular venipuncture for measurement of plasma LH concentrations from −36 hr to +72 hr of treatment. Blood samples were collected daily for measurement of plasma progesterone concentrations from 1 day before to 7 days after treatment. A Proc Mixed repeated measures analysis of variance (SAS version 9.4) was performed to compare serial data (follicle size, follicle numbers, CL size, plasma LH, plasma progesterone).

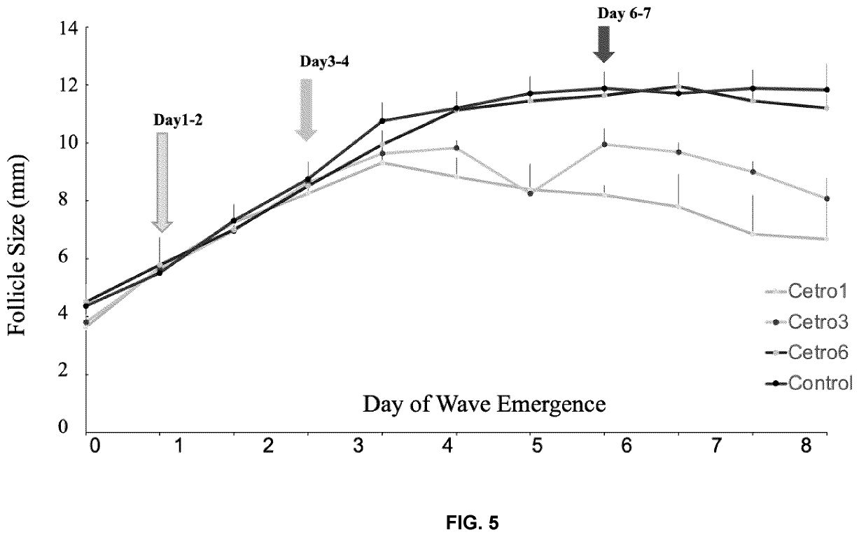

Results: The dominant follicle stopped growing in the Day 1-2 and Day 3-4 groups after cetrorelix treatment (FIG. 5). Dominant follicle size in the untreated control group indicates that the follicle has entered the static phase by Day 6-7, that is, no further increase in diameter occurred after Day 6-7. Therefore, no difference in diameter of the dominant follicle was expected between the heifers treated with cetrorelix on Day 6-7 in comparison to the untreated control group heifers.

TABLE 8
Follicle dynamics after cetrorelix treatment.

| | Control | Day 1-2 | Day 3-4 | Day 6-7 | |
|---|---|---|---|---|---|
| | n = 8 | n = 8 | n = 8 | n = 8 | P-value |
| Number of Follicles | 18.3 ± 1.9 | 19.3 ± 2.0 | 20.9 ± 2.7 | 15.8 ± 1.1 | 0.35 |
| at WE | | | | | |
| Largest Dominant | 13.2 ± 0.6^{a} | 10.3 ± 0.6^{b} | 10.6 ± 0.4^{b} | 12.9 ± 0.4^{a} | <0.01 |
| Follicle Diameter | | | | | |
| (mm) | | | | | |
| Time to Largest | 8.6 ± 1.1^{a} | 6.3 ± 0.9^{ab} | 4.6 ± 0.6^{b} | 6.8 ± 0.9^{ab} | 0.02 |
| Dominant (days) | | | | | |
| Largest Subordinate | 9.6 ± 0.6^{a} | 7.1 ± 0.5^{b} | 6.9 ± 0.4^{b} | 7.9 ± 0.6^{b} | <0.01 |
| Follicle Diameter | | | | | |
| (mm) | | | | | |
| Time to Largest | 5.0 ± 0.7 | 3.1 ± 0.6 | 4.0 ± 1.0 | 3.9 ± 0.5 | 0.39 |
| Subordinate (days) | | | | | |

There was a significant difference in the diameter of the dominant follicle between the Control group and Day 1-2 and Day 3-4 groups (Table 8). This indicated Cetrorelix caused an early regression of the follicle. There was no difference between the Control and Day 6-7 because the dominant follicle was already prepared to begin regressing.

TABLE 9
Synchrony of wave emergence after cetrorelix treatment.

| | Control | Day 1-2 | Day 3-4 | Day 6-7 | P-value |
|---|---|---|---|---|---|
| Days from treatment to next | — | 5.9 ± 0.4 | 5.8 ± 0.5 | 4.3 ± 0.6 | 0.06 |
| wave emergence | | | | | |
| Days from treatment to next | — | 9.1 ± 0.8 | 9.5 ± 0.5 | 8.3 ± 0.7 | 0.41 |
| wave's dominant follicle at 9 mm | | | | | |

Data from Table 9 indicates that the wave emergence occurred 5.3±0.3 days after the first Cetrorelix treatment irrespective of the phase of the dominant follicle. These results indicate a high degree of synchronization of wave emergence among groups.

TABLE 10
Length of follicular waves during and after treatment with cetrorelix.

| | Control | Day 1-2 | Day 3-4 | Day 6-7 | P Value |
|---|---|---|---|---|---|
| Interwave Interval | 9.1 ± 0.6^{a} | 6.9 ± 0.4^{b} | 8.9 ± 0.4^{ab} | 10.3 ± 0.6^{a} | <0.01 |
| Interovulatory Interval | 20.6 ± 0.8 | 21.5 ± 1.4 | 20.3 ± 0.4 | 19.6 ± 0.6 | 0.51 |
| Length of Wave 2 | 10.5 ± 0.8 | 9.3 ± 0.7 | 9.1 ± 0.8 | 8.3 ± 0.5 | 0.21 |

Day 1-2 group has a significantly shorter inter-wave interval. There was no difference in the ovulation interval or length of wave 2. This supports that Cetrorelix has a short half life and does not continue to affect later waves.

TABLE 11
Luteal dynamics after cetrorelix treatment.

| | Control | Day 1-2 | Day 3-4 | Day 6-7 | P Value |
|---|---|---|---|---|---|
| Largest CL Size | 23.4 ± 1.3^{a} | 19.5 ± 0.9^{b} | 20.3 ± 1.0^{ab} | 21.7 ± 0.6^{ab} | 0.05 |
| (mm) | | | | | |
| Time to Largest CL | 8.3 ± 0.9 | 8.9 ± 1.0 | 8.4 ± 0.7 | 6.6 ± 0.6 | 0.23 |
| Size (days) | | | | | |

Maximum CL diameter in Day 1-2 group was smaller than in the control group. One animal in this group had a short lived CL. The time of largest CL diameter was not affected by cetrorelix treatment

Overall, the following conclusions were drawn from this example:
i. Cetrorelix caused regression of the dominant follicle irrespective of the growth phase status
ii. A new wave emerged at 5.3±0.3 days after the first cetrorelix injection. There was a high degree of synchrony
iii. Next wave, ovulation and cycle length were not affected
iv. Next wave dominant follicle was ready for ovulation in 9 days

### Example 5: a Single Injection of Cetrorelix Induces a New Follicular Wave in Heifers

Primary objective: To test if a single 3 mg i/m injection of cetrorelix given to heifers at days following follicular wave emergence (that is, at different status of the dominant follicle) will induce synchronous wave emergence.

Hypotheses: 1) Single cetrorelix injection will cause regression of the dominant follicle. A new wave will emerge at a consistent time. 2) A high degree of ovulation synchrony will be achieved after PGF treatment. 3) Preovulatory follicle from the induced wave will be developmentally competent

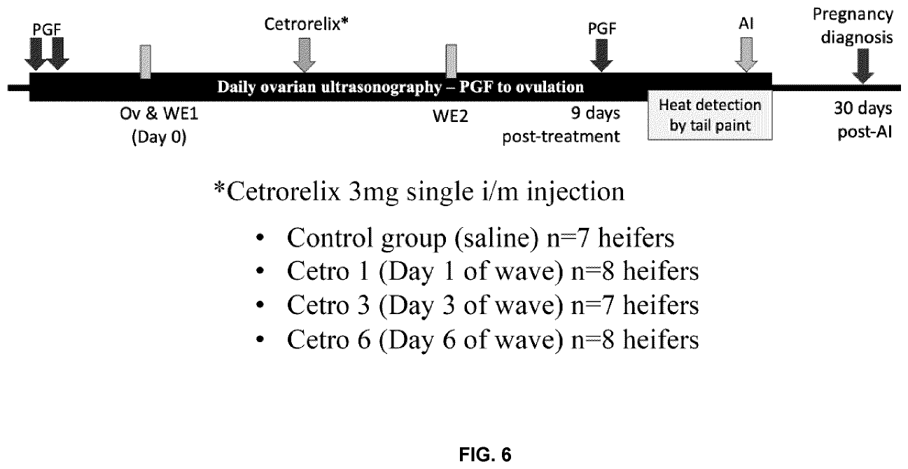

Methods: A schematic diagram of the experimental design for Example 5 is shown in FIG. 6.

Heifers on a random day of the cycle were treated with two i/m injections of prostaglandin analog and observed by transrectal ultrasonography for the day of ovulation (Defined as Day 0 of the wave). Heifers were then randomized into 4 groups (n=7 to 8 per group) and treated with normal saline (control group) or a single 3 mg i/m injection of cetrorelix on Day 1, Day 3 or Day 6 of the first follicular wave. Nine days after the saline or cetrorelix treatments, heifers were given prostaglandin analog injections and artificial insemination was done when heifers showed estrous behavior (detected by tail paint). Pregnancy diagnosis was done 30 days after artificial insemination.

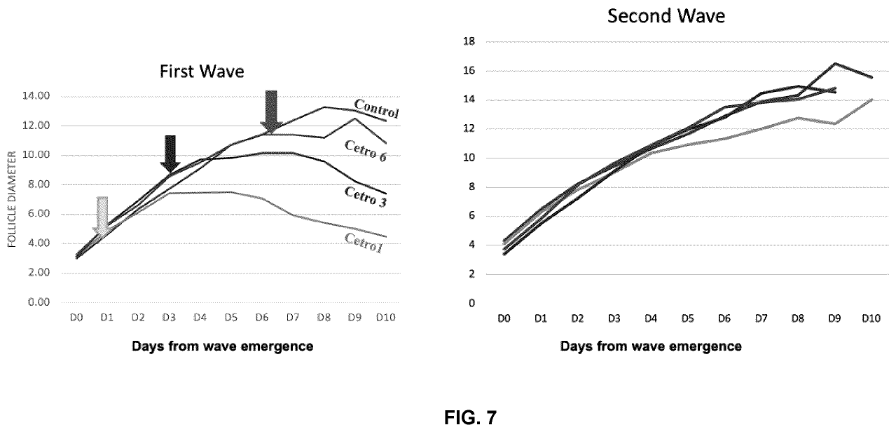

Results: Dominant follicles during the treatment wave exhibit similar growth patterns to those treated in Example 4. Dominant follicle started regression with 2 days after cetrorelix treatment (FIG. 7, left panel). The growth pattern of the dominant follicle in the next (second) wave (FIG. 7, right panel) was not affected by previous treatments.

TABLE 12
Follicle wave emergence synchrony after a single injection of cetrorelix.
Wave emergence occurred 3.5 days after the cetrorelix treatment
irrespective of the phase of the dominant follicle.

| | Control | Cetro 1 | Cetro 3 | Cetro 6 | P- |
|---|---|---|---|---|---|
| | n = 7 | n = 8 | n = 7 | n = 8 | value |
| Day of second | 10.0 ± 0.4^{a} | 4.4 ± 0.5^{c} | 7.0 ± 0.4^{b} | 9.4 ± 0.5^{a} | <0.01 |
| wave emergence | | | | | |
| Treatment to wave | — | 3.4 ± 0.5 | 4.0 ± 0.4 | 3.4 ± 0.5 | 0.56 |
| emergence (days) | | | | | |
| Days from second | 9.9 ± 0.3^{a} | 10.0 ± 0.6^{ab}* | 9.0 ± 0.4^{ab} | 8.4 ± 0.4^{b} | <0.05 |
| wave emergence | | | | | |
| to ovulation | | | | | |

TABLE 13
Ovulatory follicle and CL size after
single i/m injection of cetrorelix.

| | Control | Cetro 1 | Cetro 3 | Cetro 6 | P- |
|---|---|---|---|---|---|
| | n = 7 | n = 8 | n = 7 | n = 8 | value |
| Ovulatory | 15.9 ± 0.9 | 14.4 ± 1.0 | 15.8 ± 0.6 | 14.9 ± 0.3 | 0.57 |
| follicle size | | | | | |
| CL size on | 21.4 ± 1.0 | 21.0 ± 2.0 | 20.0 ± 1.0 | 19.5 ± 1.3 | 0.2 |
| Day 10 | | | | | |

TABLE 14
Pregnancy rate after cetrorelix treatment. Overall pregnancy
rates were similar between cetrorelix treated cattle
(n = 12/21, 57%; three cetrorelix groups combined)
and saline-treated controls (n = 3/5 = 60%).

| | Control | Cetro 1 | Cetro 3 | Cetro 6 | P-value |
|---|---|---|---|---|---|
| Pregnant/AI | 3/5 | 5/8 | 4/6 | 3/7 | 0.82 |
| | (60%) | (62%) | (66%) | (42%) | |

The following conclusions were drawn from this example:
i. A new wave emerged at 3.5±0.4 days after a single 3 mg cetrorelix i/m treatment. There was no effect of the status of the dominant follicle and follicular wave on the interval between treatment and wave emergence.
ii. There was a high degree synchrony for both wave emergence and ovulation.
iii. The oocytes produced in the second (induced) wave were fertile.

### Example 6: Effect of Cetrorelix During the Luteal Phase (High Progesterone) Versus Preovulatory Phase (Low Progesterone) in and Heifers

Specific Objectives: Compare the growth pattern and ovulatory potential of the dominant follicle in cattle following treatment with Cetrorelix during the luteal phase (Day 5-6 of a wave during a period with a functional corpus luteum) and the preovulatory phase (Day 5-6 of wave during a period with a regressing corpus luteum).

Hypotheses: 1. Cetrorelix will cause regression of both the mid-static phase dominant follicle and the preovulatory stage dominant follicle and 2. the dominant follicle in animals treated with Cetrorelix will not ovulate in response to exogenous LH (hCG).

Experimental design: The study was conducted on pubertal Hereford-cross beef heifers (n=24) at the U. Sask. Livestock and Forage Centre of Excellence (LFCE) using a 2×2 factorial design (Table 15). Initial transrectal ultrasonography was performed to confirm post-pubertal or post-partum cyclicity by the presence or absence of a corpus luteum (CL). Animals with a CL were weighed and treated with prostaglandin F2a analog as in Example 4 and monitored by ultrasonography to detect ovulation. Day of ovulation was considered the day of wave emergence (defined as Day 0).

TABLE 15
Experimental design (2 × 2 factorial design)

| | Luteal-Phase | Preovulatory Phase |
|---|---|---|
| Control | n = 6 heifers | n = 6 heifers |
| Treatment | n = 6 heifers | n = 6 heifers |

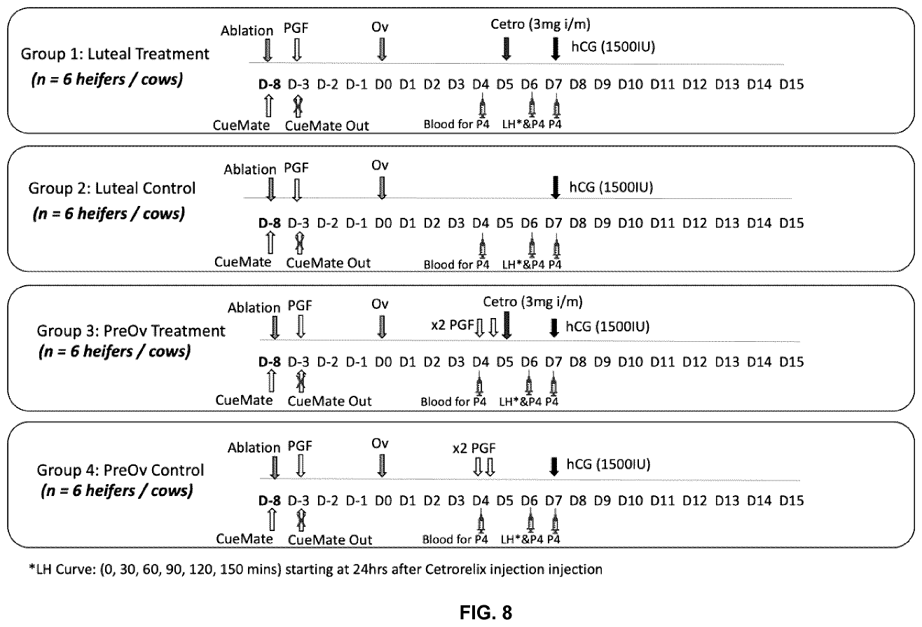

On the day of ovulation, heifers were randomized into 4 groups. Those in the preovulatory groups were given prostaglandin on Days 4 and 4.5 of the wave while those in the luteal groups were allowed to maintain the CL (i.e., no prostaglandin treatment). Treatments was given as outlined in Table 16 and schematic time line FIG. 8. Cetrorelix groups were given 3 mg cetrorelix i/m on Day 5. All animals were treated with hCG (Chorulon, 1500 IU, im) on Day 7 of the wave (i.e., 48 hr after Cetrorelix/saline treatment) to determine the ovulatory potential of the dominant follicle.

TABLE 16
Treatment Protocol:

| | Day of the Follicular Wave | | | |
|---|---|---|---|---|
| | (Day 0 = Wave Emergence) | | | |
| | Luteal Phase | | Pre-ovulatory Phase | |
| Treatment Group | Day 5 | Day 7 | Day 5 | Day 7 |
| Luteal Control | 3 ml | 1500 IU | | |
| (n = 6 heifers) | saline | hCG | | |
| Luteal Treatment | 3 mg | 1500 IU | | |
| (n = 6 heifers) | Cetrorelix | hCG | | |
| PreOv Control | | | 3 ml | 1500 IU |
| (n = 6 heifers) | | | saline | hCG |
| PreOv Treatment | | | 3 mg | 1500 IU |
| (n = 6 heifers) | | | Cetrorelix | hCG |

Blood samples were taken 24 hr after cetrorelix or no treatment (control groups) for measurement of plasma concentrations of LH and progesterone. Data was analyzed in a similar manner to Example 4.

Results: ovulations occurred in all 4 groups (cetrorelix and control; low and high progesterone) after treatment with hCG indicating that, in contrast to proposed hypothesis 2, the dominant follicle under high progesterone environment and the pre-ovulatory follicle in low-progesterone environment maintain the ovulatory potential for at least 48 hr after cetrorelix treatment and have not yet regressed, i.e. the follicles respond to exogenous hCG (LH) by ovulating. Cetrorelix group heifers ovulated on Day 9 or 10 (3 to 4 days after cetrorelix treatment) leading to start of a new follicular wave. This time duration matches with the previous experiment indicating cetrorelix treatment followed by LH or hCG can be also be an effective way to start a synchronous follicular wave emergence.

### Example 7: Effect of a Single Injection of Cetrorelix Given on Random Day of Follicular Wave (and Estrous Cycle) in Heifers

Primary objective: Examine the effect of cetrorelix treatment given at random day of the cycle on follicle wave synchronization, ovulation synchronization and pregnancy rate after fixed-time artificial insemination in heifers

Hypotheses: 1) Cetrorelix treatment given on a random day of the cycle will induce a new wave at a consistent time, 2) Cetrorelix protocol results in synchronization of ovulation and acceptable pregnancy rate after fixed-time artificial inseminations and 3) This steroid-free ovulation synchronization protocol is as effective as estradiol-based fixed-time AI protocol.

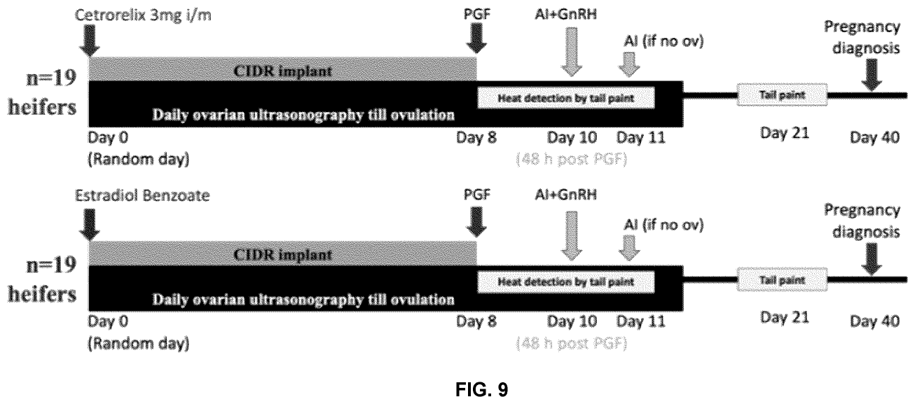

Methods: A schematic diagram of the experimental design for Example 7 is shown in FIG. 9.

This experiment was conducted on 38 mature Angus heifers at the Federal University of Santa Maria in Brazil. Heifers on a random day of the cycle (defined as Day 0) were divided into 2 groups (n=19 per group) and treated with a single i/m injection of 3 mg cetrorelix or with estradiol benzoate (1 mg i/m). A progesterone-releasing device (CIDR) was placed in the vagina of heifers at this time. Eight days after the cetrorelix or estradiol treatments, heifers were given 500 ug cloprostenol (prostaglandin analog) i/m injections and the CIDR device was removed. Artificial insemination (AI) was done when heifers showed estrous behavior (detected by tail paint). Heifers were given 250 ug gonadorelin acetate (GnRH analog) i/m injection at the time of artificial insemination. If ovulation did not occur within 24 hr of GnRH treatment, a second AI was performed. Pregnancy diagnosis was done 29-30 days after artificial insemination.

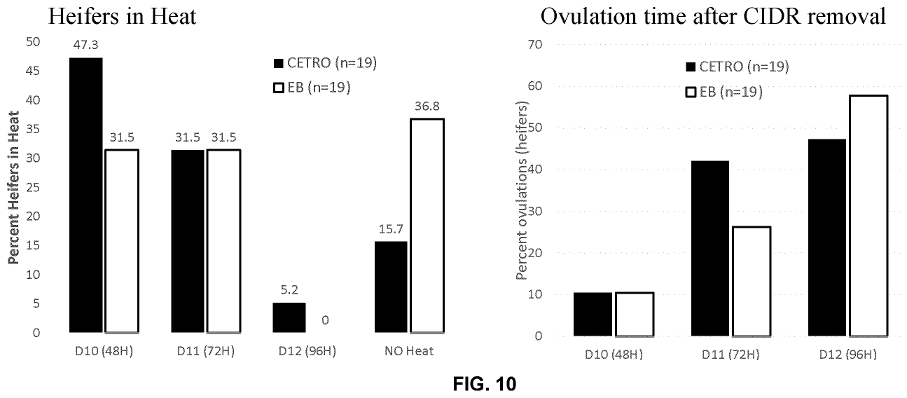

Results: A greater proportion of heifers showed estrous behavior after cetrorelix treatment than the estradiol-progesterone treatment (FIG. 10, left panel). The majority of ovulations occurred between 72 to 96 hour after CIDR removal in both treatment groups (FIG. 10, right panel).

TABLE 17
Pregnancy rate after cetrorelix compared to the estradiol-progesterone
protocol. Early ovulations were detected at the time of artificial
insemination in two heifers of cetrorelix group and two heifers
of the estradiol-progesterone group.

| | Cetrorelix | Estradiol | P-value |
|---|---|---|---|
| Pregnant/Total | 15/19 (79%) | 13/19 (68%) | 0.71 |
| Pregnant/Single AI | 8/10 (80%) | 6/7 (86%) | 1.0 |
| Pregnant/Double AI | 7/9 (78%) | 7/12 (58%) | 0.64 |

The Cetrorelix protocol results in synchronization of ovulation and numerically higher pregnancy rate (79%) after fixed-time artificial inseminations in angus heifers than the pregnancy rate after applying the existing standard estradiol-progesterone protocol (68%); albeit, there was no statistically difference between the two treatments. Pregnancy rates were not different between heifers that were inseminated once or required a second insemination.

Following are the conclusions of this study:
i. Based on the ovulation and pregnancy data, treatment with a GnRH antagonist, Cetrorelix, caused regression of the dominant follicle regardless of status of the follicular wave
ii. Based on the ovulation and pregnancy data, Cetrorelix treatment led to emergence of a new follicular wave at a consistent interval from the beginning of treatment
iii. The majority (90%) of ovulations occurred between 72 to 96 hr after prostaglandin injection in the cetrorelix protocol and heifers exhibited improved estrous behaviour after cetrorelix protocol compared to the estadiol-progesterone based protocol.
iv. Pregnancy rates after cetrorelix protocol in beef heifers (79%) were slightly better than those after estradiol-progesterone protocol (68%) but did not differ statistically.
   Example 8: Field Trial to Determine the Conception (Pregnancy) Rate after Single Injection of Cetrorelix Given on Random Day of Follicular Wave and Estrous Cycle in Post-Partum Beef Cows

Objective: The objective of the study was to compare conception rate after single fixed-time artificial insemination in multiparous cows after cetrorelix versus estradiol synchronization protocol

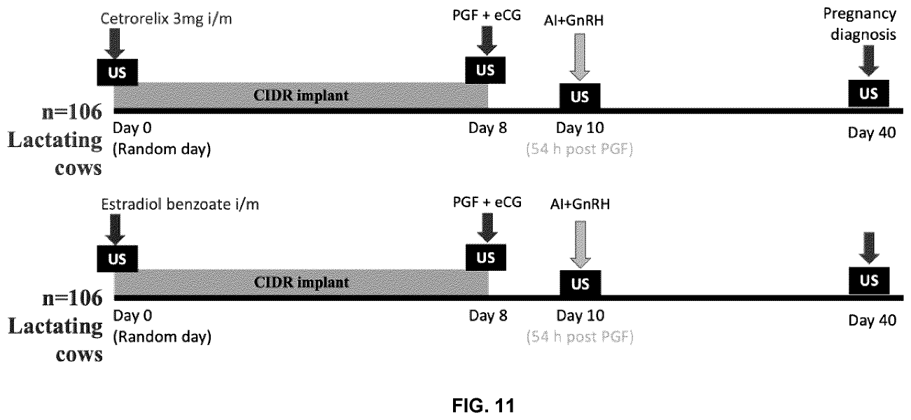

Methods: A schematic diagram of the experimental design for Example 8 is shown in FIG. 11.

This field trial was conducted by a faculty member of the Federal University of Santa Maria at a client beef farm in Brazil in December 2022. A total of 216 Brahman x Angus cross beef post-partum cows were used for this experiment. Cows were divided into two treatment groups (n=106 per group) and were either given cetrorelix 3 mg i/m or estradiol benzoate on a random day of the cycle (defined as Day 0). An intravaginal progesterone device (CIDR) was placed in all cows on Day 0. The CIDR devices were removed on Day 8 and animals were given prostaglandin F2a analog and equine choriogonadotropin (eCG). Fixed-time artificial insemination was done 54 hrs after CIDR removal and animals were given GnRH at this time. Pregnancy diagnosis was performed on Day 30 after artificial insemination. Transrectal ultrasound examinations were performed on Day 0, Day 8, Day 10 and Day 40.

Results: The results of the study are shown in Table 18 and 19.

TABLE 18
Size on the dominant follicle on the day of CIDR removal (Day
8) and the day of fixed-time artificial insemination (Day 10).

| | Day 8 (at PGF) | Day 10 (at AI) |
|---|---|---|
| Cetrorelix | 11.04 ± 0.48 | 12.72 ± 0.65 |
| Estradiol benzoate | 8.60 ± 0.39 | 11.38 ± 0.56 |

Dominant follicle was larger in the cetrorelix group than the estradiol group on the day of prostaglandin treatment (day of CIDR removal) and on the day of fixed-time artificial insemination. Cows had greater amount of mucous discharge and display of estrus behavior in the cetrorelix group than in the estradiol group

TABLE 19
Conception rates at 30 days after fixed-time artificial insemination.

| | Pregnant/AI | Percent |
|---|---|---|
| Cetrorelix | 46/108* | 42.6% |
| Estradiol benzoate | 48/106* | 45.6% |

*Chi Square P-value = 0.69

Conception (pregnancy) rates did not differ between the cetrorelix protocol (42.6%) and the estradiol-based protocol (45.6%). It is noteworthy that on the day of fixed-time artificial insemination, ambient temperature was more than 40° C. leading to extreme heat stress which might have affected the overall pregnancy rate in both groups. Nevertheless, there is no difference in pregnancy rate between the two groups indicating that cetrorelix protocol is as effective as estradiol protocol even under extreme heat stress conditions.

Conclusions: The overall conclusions of this study were:
i. The dominant preovulatory follicle in the cetrorelix group had size-advantage over that in the estradiol-based protocol on the day of CIDR removal and on the day of fixed-time artificial insemination.
ii. Cetrorelix protocol was as effective as the estradiol-based protocol when comparing pregnancy rates (that is, conception rates did not differ between the Cetrorelix-based protocol and the estradiol-based protocol. Cows displayed better estrous behaviour in the Cetrorelix protocol.

### Example 9: Effect of Four GnRH Antagonists on LH Secretion of Bovine Pituitary Cells In Vitro

Objective: To compare the efficacy of four GnRH antagonist (Cetrorelix, degarelix, abarelix and relugolix) on blocking the GnRH-mediated LH release from the bovine gonadotrophs using the in vitro culture of bovine pituitaries.

Hypothesis: Pituitary cells treated in vitro with GnRH antagonists will have lower levels of LH secretion than untreated control group after cells are stimulated with GnRH.

Methods: The heads of mature, non-pregnant cows (n=4) were collected from a local abattoir and the pituitary glands were dissected within 60 minutes of slaughter. Immediately following the dissection, the pituitaries were submerged into ice-cold collection medium. The pituitaries were washed 3 times in collection media and the neurohypophysis was dissected from the adenohypophysis. Using a scalpel, the adenohypophysis (anterior pituitary) was sliced into 1 mm×1 mm pieces and transferred to a 50 mL falcon tube containing DMEM medium with 0.5% BSA. The tissue pieces were washed by manually agitating the tube for 1 minute. The tissue pieces were allowed to settle to the bottom of the tube, then the supernatant was discarded. The macerated pieces were transferred to a 15 mL falcon tube with the dissociation medium and incubated at 37° C. for 60 minutes. Every 10 minutes the tube was vortexed for 1 minute to ensure the tissue was completely dissociated. The dissociation media was inactivated using DMEM with 10% SFB then manually agitating the tube for 1 minute. The dissociated cells were placed in a 40 μm cell strainer to remove the undigested tissue and placed into two 15 mL falcon tubes. The cells were centrifuged at 200×g for 10 minutes and the supernatant was discarded. The cells were washed three times by resuspending in DMEM solution with red blood cell lysing buffer followed by centrifuged at 200×g for 10 minutes. Then the cells were washed with DMEM solution and centrifuged at 200×g for 10 minutes. Following the last centrifugation, the cells were resuspended in DMEM, and the trypan blue test was used to determine the cell count and assess viability. DMEM with 10% fetal bovine serum (FBS) was treated with 10% charcoal-dextran and passed through a 0.2 μm filter. The cells were then plated in a 24-well tissue culture plate with a concentration of 1×10⁶ viable cells per well and incubated at 37° C. for 24 hours. The following day, to starve the cells, the culture media was replaced with DMEM without FBS and left to incubate for another 24 hours.

Treatments: Based on the results from the pilot study, the selected concentration for Cetrorelix (cetrorelix acetate) was 300 μg. The cetrorelix was dissolved in trace levels of DMSO then dissolved in ultrapure water. To standardize the treatments, the nanomole concentration of each GnRH antagonist were matched with the cetrorelix nanomole concentration. The final concentration used for further experiments were: 300 μg for cetrorelix, 340 μg for Degarelix (Degarelix acetate), 300 ug for Abarelix (Abarelix acetate). Both degarelix and abarelix were dissolved ultrapure water. The equivalent Relugolix nanomole concentration was 130 μg which was dissolved in methanol. Treatment cells were preincubated for 60 minutes with either Cetrorelix, Degarelix, Relugolix or Abarelix. Following the preincubation, the cells were incubated for 30 minutes with either 1 nM or 10 nM of GnRH (Gonadorelin, Fertagyl, Merck Animal Health). GnRH doses were based on pituitary study by Paolicchi et al. 1999). The media were collected and centrifuged at 200×g for 10 minutes to remove the cells. Positive (Control) group was only treated with 1 nM and 10 nM of GnRH to determine the maximum LH secretion. LH concentrations in the culture medium was measured by a commercial bovine specific ELISA kit and reported as mIU/ml of medium.

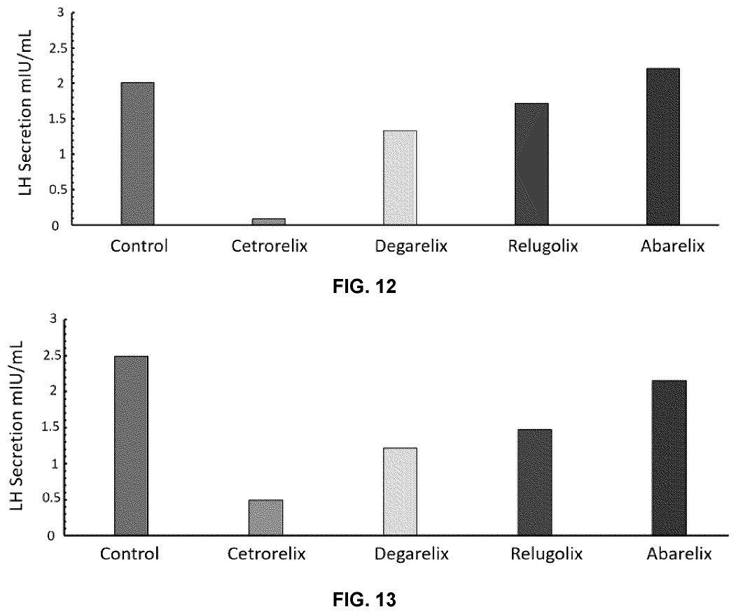

Results: The dissociation procedure yielded 20-50×10⁶ cells per pituitary gland (n=4) and had a cell viability >80%. It is worth noting that the Cetrorelix is a fast-acting peptide, Relugolix is a fast-acting non-peptide, and Degarelix is a slow-acting peptide. The results of the study are illustrated in FIGS. 12 and 13 and shown in Tables 20 and 21.

TABLE 20
LH concentration in medium from pituitary cells
in vitro when challenged with 1 nM GnRH

| | LH conc |
|---|---|
| GnRH Antagonist | (mIU/mL) |
| Control | 2.01 |
| Cetrorelix | 0.09 |
| Degarelix | 1.33 |
| Relugolix | 1.72 |
| Abarelix | 2.21 |

TABLE 21
LH concentration in medium from pituitary cells
in vitro when challenged with 10 nM GnRH.

| | LH conc |
|---|---|
| GnRH Antagonist | (mIU/mL) |
| Control | 2.49 |
| Cetrorelix | 0.50 |
| Degarelix | 1.21 |
| Relugolix | 1.47 |
| Abarelix | 2.15 |

A decrease in LH secretion as compared to the positive control was expected. The Cetrorelix was most effective in blocking the GnRH-induced LH secretion at both 1 nM and 10 nM GnRH stimulation), that is cetrorelix groups in FIGS. 12 and 13 and Tables 20 and 21 had the lowest LH levels for each concentration of GnRH given. The Degarelix and Relugolix both showed a degree of LH suppression as they had lower LH levels than the control. At the concentration used, Abarelix had no effect on the LH suppression at 1 nM GnRH challenge but resulted in modest decrease in LH concentration when challenged with 10 nM of LH. Dose-dependent increase in LH secretion was recorded when results of 1 nM and 10 nM GnRH challenge were compared between the same drug. Furthermore, the ranking of the drugs remained unchanged between 1 nM and 10 nM GnRH challenge.

Conclusions: Overall conclusion of the study are:
i. Cetrorelix is a fast-acting GnRH-anatgonist peptide, relugolix is a fast-acting non-peptide, and degarelix is a slow-acting peptide. These three GnRH antagonists lead to blockage of GnRH-induced LH secretion compared to the control at low-dose (1 nM) and high-dose (10 nM) of GnRH in vitro incubation.
ii. Abarelix failed to block LH secretion at 1 nM of GnRH challenge but showed some modest blocking effect at 10 nM of GnRH challenge. It is likely that abarelix requires a higher nanomolar concentration to be effective at reducing LH levels.
iii. Cetrorelix was the most effective antagonist at reducing LH levels.
iv. There was a difference in the blocking efficiencies of the GnRH antagonists. Similar ranking of the cetrorelix, degarelix, relugolix, and abarelix at 1 nM and 10 nM GnRH challenge indicate the consistency of the response.
v. The results of this experiment confirm that the underlying mechanism of action (i.e. ability to block LH secretion from pituitary gland) is similar between the four tested GnRH antagonists but their efficiency of blockage at a fixed nanomolar concentration differed between the drugs. Therefore, based on these results, degarelix, relugolix, and abarelix and other GnRH antagonists are predicted to have similar in vivo effects as shown for Cetrorelix, for example on synchronization of follicular wave emergence, however, each drug will require calibration of effective dose.

### Example 10: The Effect of Four GnRH Antagonists (Cetrorelix, Degarelix, Relugolix, Abarelix) on GnRH-Induced Plasma LH Levels and Ovulation in Cattle

Objective: To identify the effect of GnRH antagonists on the plasma LH levels and ovulation of heifers following an injection of GnRH.

Hypotheses: 1) GnRH antagonists (cetrorelix, degarelix, relugolix, abarelix) will prevent an LH rise irrespective of administration of GnRH. 2) Animals treated with GnRH antagonists will not ovulate.

Methods: Pubertal heifers (n=10) with a functional CL were selected at the Federal University of Santa Maria. Heifers were administered an intramuscular (i/m) injection of prostaglandin F2a analog (PGF; Lutalyse 5 ml or Estrumate 2 ml) to induce CL regression. Heifers were randomly placed into 4 treatment groups at the time of PGF injection: Trt1 (n=2, 3 mg of Abarelix); Trt2 (n=2, 3 mg of Degarelix), Trt3 (n=2, 3 mg of Relugolix) and Trt 4 (n=4; Cetrorelix 3 mg). Heifers were treated with these GnRH antagonists 1.5 days (36 hr) after the PGF injection and the GnRH antagonist treatment time point was defined as −60 mins. Heifers were treated with GnRH (2 mg of Fertagyl i/m) at 0 mins to induce ovulation. Jugular venipuncture plasma samples were collected every 60 minutes for 3 hours (from −60 min to 120 minutes, with t=0 min when GnRH is administered). Plasma samples were kept at 4C overnight and LH analysis was performed the next day using a bovine-specific ELISA commercial kit. Transrectal ultrasonography was conducted to the time of GnRH injection (Time 0 hr) and 2 days later (48 hr after GnRH injection) to record the size of dominant follicle and to detect ovulation after treatment.

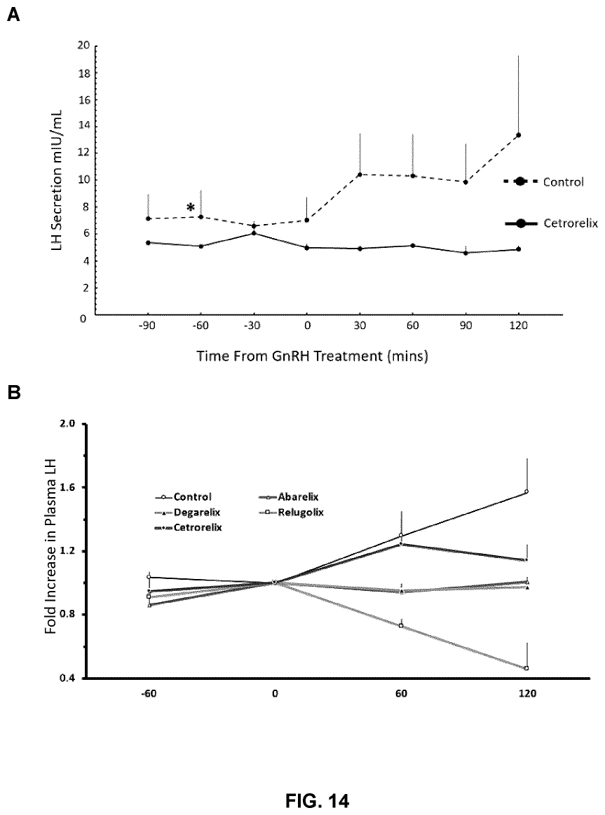

Results: FIG. 14A shows a proof-of-concept example using cetrorelix. GnRH injection was given at Time 0 min: untreated control showed 1.5-fold increase in plasma LH concentration within 30 minutes while LH levels remained unchanged in heifers pre-treated with cetrorelix 60 minutes before the GnRH injection. Table 22 shows the plasma LH concentration of individual animals at −60 hr (time of GnRH antagonist injection), 0 min (GnRH challenge), and 60 min and 120 minute after GnRH injection.

TABLE 22
Plasma LH concentration (mIU/ml) after GnRH-challenge in heifers
pre-treated with Abarelix, Degarelix, Relugolix, Cetrorelix
and control (no pre-treatment). Data from individual heifers
and mean ± standard error per group are provided. Time
0 min = time of GnRH injection. Injections of GnRH antagonists
are given 60 mins before GnRH injection (at Time −60 min).

| | Plasma LH concentration (mIU/ml) at | | | | |
|---|---|---|---|---|---|
| | Animal | −60 | 0 | 60 | 120 |
| Group | ID | minute | minute | minute | minute |
| Control | SB | 9.46 | 9.95 | 10.36 | 13.94 |
| Control | 1602 | 12.33 | 10.98 | 13.79 | 13.94 |
| Control | 2104A | 5.32 | 5.34 | 7.19 | 7.42 |
| Control | 2017A | 9.25 | 8.71 | 13.42 | 19.30 |
| | Mean | 9.09 | 8.75 | 11.19 | 13.65 |
| | SEM | 1.44 | 1.23 | 1.54 | 2.43 |
| Abarelix | 2118 | 8.65 | 8.98 | 8.96 | 9.32 |
| Abarelix | 2110 | 10.21 | 13.54 | 12.02 | 13.14 |
| | Mean | 9.43 | 11.26 | 10.49 | 11.23 |
| | SEM | 0.78 | 2.28 | 1.53 | 1.91 |
| Degarelix | 3 | 5.54 | 5.37 | 4.87 | 5.25 |
| Degarelix | 2202 | 10.74 | 12.41 | 12.32 | 11.98 |
| | Mean | 8.14 | 8.89 | 8.60 | 8.62 |
| | SEM | 2.60 | 3.52 | 3.73 | 3.37 |
| Relugolix | 2104 | 30.96 | 33.09 | 22.66 | 9.71 |
| Relugolix | 951 | 5.99 | 6.75 | 5.22 | 4.23 |
| | Mean | 18.48 | 19.92 | 13.94 | 6.97 |
| | SEM | 12.49 | 13.17 | 8.72 | 2.74 |
| Cetrorelix | 2108 | 24.29 | 18.10 | 18.33 | 23.74 |
| Cetrorelix | 2107 | 12.03 | 12.23 | 22.72 | 15.04 |
| Cetrorelix | 2108A | 5.24 | 5.29 | 5.12 | 4.60 |
| Cetrorelix | 2118A | 4.96 | 4.66 | 5.15 | 5.32 |
| | Mean | 11.63 | 10.07 | 12.83 | 12.18 |
| | SEM | 4.53 | 3.18 | 4.53 | 4.53 |

As there was large individual variation in plasma LH concentration before treatment with LH antagonists in Table 22, data were normalized to the time of GnRH injection (considered as 1) and fold-change for each group is presented in FIG. 14B. In the positive control group (i.e., without any GnRH antagonist treatment), LH concentrations increased 1.57 fold in 2 hours, while animals pre-treated with Abarelix, Degarelix, Relugolix or Cetrorelix did not register an increase in LH when challenged with GnRH indicating that all four tested GnRH antagonists drugs were effective in blocking GnRH-induced LH secretion in vivo. Table 23 shows the size of the dominant follicles at the time of GnRH treatment and 48 hr later as well as if ovulation was detected by 48 hr after GnRH treatment. All cattle pre-treated with either Abarelix, Degarelix, Relugolix or Cetrorelix failed to ovulate by 48 hr after GnRH injections while both the positive control group animals (i.e., those given GnRH alone) ovulated by 48 hr.

TABLE 23
Dominant follicle diameter of individual heifers in each group
at the time of GnRH treatment and 48 hr later. Last column lists
if ovulation occurred (Yes/No) within 48 hr of treatment.

| | | Dominant follicle diameter (mm) | | |
|---|---|---|---|---|
| Treatment | | at GnRH | 48 hr after | |
| Group | Animal ID | injection | GnRH Injection | Ovulation |
| Control | SB | 13.2 | — | Yes |
| Control | 1602 | 12.3 | — | Yes |
| Abarelix | 2118 | 14.4 | 14 | No |
| Abarelix | 2110 | 13.6 | 12 | No |
| Degarelix | 3 | 14 | 12 | No |
| Degarelix | 2202 | 15.1 | 13.2 | No |
| Relugolix | 2104 | 9.8 | 10 | No |
| Relugolix | 951 | 12.1 | 10 | No |
| Cetrorelix | 2108 | 7.5 | 9.8 | No |
| Cetrorelix | 2107 | 10.5 | 12 | No |

Conclusions: Both hypotheses are supported. All four GnRH antagonists (abarelix, degarelix, relugolix and cetrorelix) block the GnRH-induced LH release in cattle and prevent GnRH-induced ovulations indicating that any of these GnRH antagonists drugs can be used in the breeding management protocols described herein.

### Example 11: Effect of GnRH Antagonist on Ovarian Follicular Dynamics in Cows

Objective: To test the effect of administration of other GnRH antagonists (selected drugs including but not limited to degarelix, abarelix, ganirelix, antide, relugolix, elagolix, or acyline) in the development of follicular wave when injected at day 1 and 2, day 3 and 4, or day 5 and 6 after follicular wave emergence.

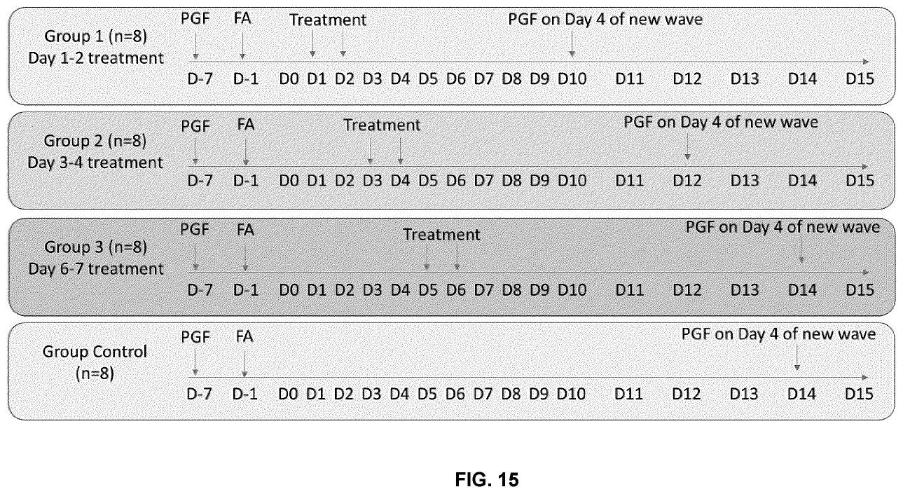

Methods: A schematic diagram of the experimental design for Example 11 is shown in FIG. 15

Experimental design is very similar to Example 4, except that PGF is used to induce ovulation before the start of the experiment, and FA (Follicle ablation) is done 7 days after PGF (approx. 3-4 days after ovulation).

Two i/m injections of treatment (24 hr apart) will be used-one or more of the following drugs are tested in parallel: degarelix, abarelix, ganirelix, antide, relugolix, elagolix, or acyline. Treatment is given on Day 1-2, or 3-4 or 5-6 of the wave.

PGF is given at Day 4 of new (post-treatment) wave emergence to cause ovulation for re-use of the animals or for breeding (i.e., not part of the experimental design)

The following endpoints will be measured:
Daily diameter measurement of the largest (dominant) follicle present at the time treatment administration (to regression or ovulation)
Day of new wave emergence after treatment.
Daily diameter measurement of the dominant follicle of the new wave after treatment
Day of ovulation after treatment
Day of ovulation after wave emergence
Daily CL diameter
Doppler echotexture for CL (visual scoring from Grade 1 to 4): Grade 1=functional diestrus CL at PGF injection; Grade 2=24 hrs after PGF; Grade 3=48-72 hrs after PGF; Grade 4=One day after ovulation detection (metestrus). Also use CL diameter in association with grading scheme
Blood samples 48 hrs after end of GnRH antagonist treatment for progesterone

Expected Results: Follicle wave emergence is synchronized by the GnRH antagonists tested.

### Example 12: Comparison of Different GnRH Antagonists

Objective: To compare the effect of different GnRH antagonists on follicular dynamics in cows

The effects of other GnRH antagonists (e.g. acyline, antarelix/teverelix, degarelix, ganirelix, antide, relugolix, elagolix, abarelix, prazarelix, ramorelix, antide, detirelix, ozarelix, linzagolix, opigolix, sufugolix and/or A-75998) are tested in parallel for comparison to Cetrorelix.

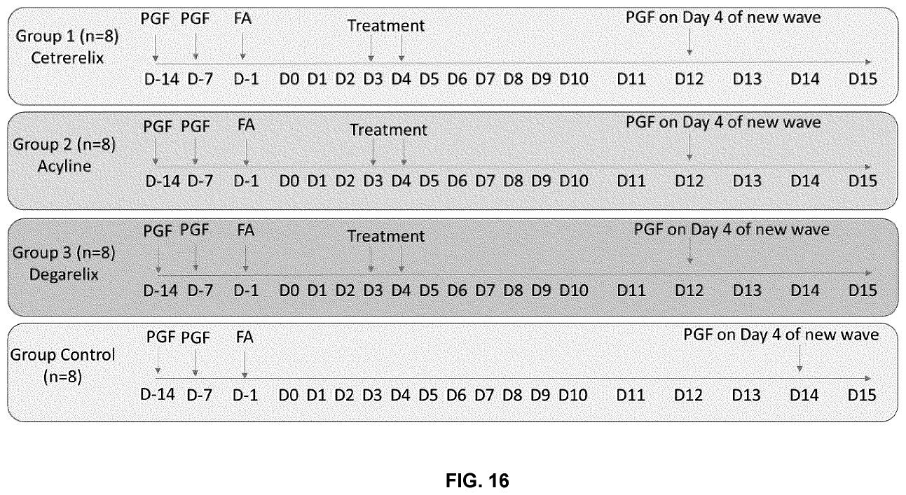

Methods: A schematic diagram of the experimental design for Example 12 is shown in FIG. 16. Experimental design and endpoints are very similar to Example 11, except that only one timepoint of administration (Day 3-4 of wave) will be tested for treatment with the drugs.

Expected Results: Cetrorelix, acyline, antarelix/teverelix, degarelix, ganirelix, antide, relugolix, elagolix, abarelix, prazarelix, ramorelix, antide, detirelix, ozarelix, linzagolix, opigolix, sufugolix and/or A-75998 all synchronize follicular wave emergence, but the duration between treatment and day of FWE may be different among the tested drugs.

### Example 13: Comparison of Single Vs Double Doses

Objective: To compare the effect of administration of single versus two injections (24 hrs apart) of treatment (Cetrorelix versus Degarelix/other GnRH antagonist) on follicular dynamics in heifers.

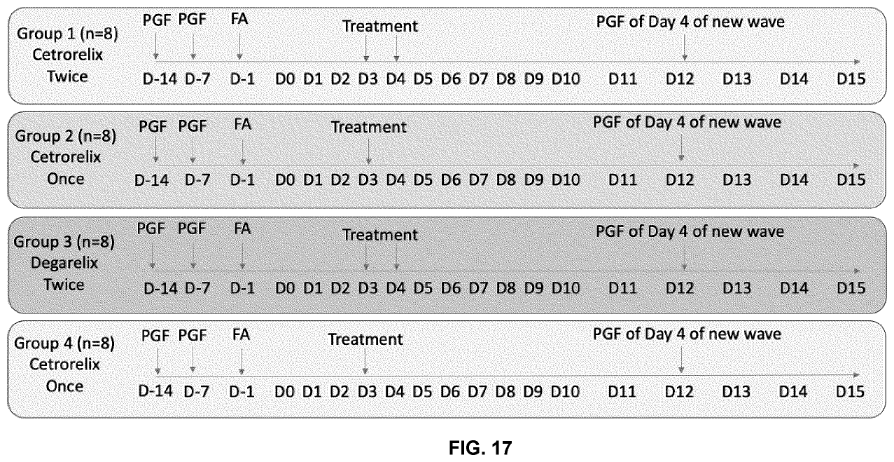

Methods: A schematic diagram of the experimental design for Example 13 is shown in FIG. 17. Experimental design and endpoints are very similar to Example 11, except that the number of administrations (one vs two) of GnRH antagonist given at day 3 or day 3-4, respectively, are compared.

Expected Results: Administration of a single dose or two dose series of GnRH antagonist synchronizes FWE, but the duration between treatment and day of FWE may be different.

### Example 14: Cetrorelix Dose Optimization in (Beef) Cattle

Objective: To determine the minimal effective i/m dose of single injection of cetrorelix to synchronize wave emergence and ovulation.

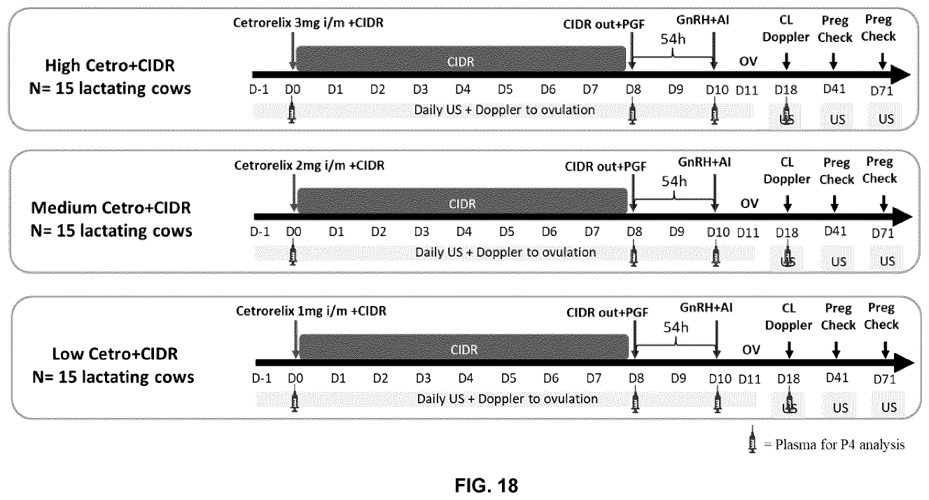

Methods: A schematic diagram of the experimental design for Example 14 is shown in FIG. 18. Lactating beef cows on a random day of the cycle (defined as Day 0) are divided into 3 groups (n=15 per group) and treated with a single i/m injection of 1) 3 mg cetrorelix; 2) 2 mg cetrorelix; or 3) 1 mg cetrorelix. A progesterone-releasing device (CIDR) is placed in the vagina of heifers at this time. Eight days after the cetrorelix treatment, cows are given prostaglandin analog injections and the CIDR device is removed. Animals are tail-painted or Estrotect patched at CIDR removal, and checked daily until ovulation. Fixed-time artificial insemination is done 54 hrs after CIDR removal and animals are given GnRH at this time. Pregnancy diagnosis is done 30 and 60 days after artificial insemination.

The following endpoints are measured:
Duration between Cetrorelix injection and wave emergence
Synchrony of wave emergence.
Diameter of dominant follicle at the time PGF and AI
Duration between Cetrorelix and ovulation
Ovulation rates and synchrony of ovulation
Corpus luteum blood flow and plasma progesterone on Day 7 after ovulation
Plasma progesterone levels on Day of Cetrorelix treatment, PGF and AI
Conception rates on Day 30 and Day 60 after AI

Expected Results: The minimum effective i/m dose of cetrorelix is between 1 mg and 2 mg.

While the present application has been described with reference to what are presently considered to be the preferred examples, it is to be understood that the application is not limited to the disclosed examples. To the contrary, the application is intended to cover various modifications and equivalent arrangements included within the spirit and scope of the appended claims. The scope of the claims should not be limited by the preferred embodiments and examples, but should be given the broadest interpretation consistent with the description as a whole.

All publications, patents and patent applications are herein incorporated by reference in their entirety to the same extent as if each individual publication, patent or patent application was specifically and individually indicated to be incorporated by reference in its entirety.

TABLE 24
Suitable GnRH Antagonists.
GnRH Antagonists: Peptides

1. Generic Name: Cetrorelix

| | |
|---|---|
| a. | Commercial Name: Cetrotide |
| b. | IUPAC Structure: N-acetyl-3-(2-naphthyl)-D-alanyl-4-chloro-D-phenylalanyl-3- (3- |
| | pyridyl)-D-alanyl-L-seryl-L-tyrosyl-D-citrullyl-L-leucyl-L-arginyl-L-prolyl-D- |
| | alaninamide |

2. Generic Name: Abarelix

| | |
|---|---|
| a. | Commercial Name: Plenaxis |
| b. | IUPAC Structure: N-acetyl-3-(2-naphthyl)-D-alanyl-4-chloro-D-phenylalanyl-3- (3- |
| | pyridyl)-D-alanyl-L-seryl-N-methyl-L-tyrosyl-D-asparagyl-L-leucyl-N6-isopropyl-L- |
| | lysyl-L-prolyl-D-alaninamide |

3. Generic Name: Degarelix

| | |
|---|---|
| a. | Commercial Name: Firmagon |
| b. | IUPAC Structure: N-acetyl-3-(2-naphthyl)-D-alanyl-4-chloro-D-phenylalanyl- 3-(3- |
| | pyridyl)-D-alanyl-L-seryl-4-((S)-dihydroorotamido)-L-phenylalanyl-4-ureido-D- |
| | phenylalanyl-L-leucyl-N6-isopropyl-L-lysyl-L-prolyl-D-alaninamide |

4. Generic Name: Ganirelix

| | |
|---|---|
| a. | Commercial Name: Antagon, Orgalutran |
| b. | IUPAC Structure: L-arginyl-D-tryptophyl-N-methyl-L-phenylalanyl-D- tryptophyl-L- |
| | leucyl-L-methioninamide |

5. Generic Name: Teverelix, Antarelix

| | |
|---|---|
| a. | Commercial Name: |
| b. | IUPAC Structure: N-acetyl-3-(2-naphthyl)-D-alanyl-4-chloro-L-phenylalanyl- 3-(3- |
| | pyridyl)-D-alanyl-L-seryl-L-tyrosyl-D-homocitrullyl-L-leucyl-N6-isopropyl-L-lysyl-D- |
| | prolyl-DL-alaninamide |

6. Generic Name: Prazarelix

| | |
|---|---|
| a. | Commercial Name: |
| b. | IUPAC Structure: 2-(N-((R)-2-((R)-2-acetamido-3-(naphthalen-2-yl) propanamido)- |
| | 3-(4-chlorophenyl)propanoyl)-D-tryptophyl-L-seryl-L-tyrosyl-O-((2R,3R,4R,5R,6S)- |
| | 3,4,5-trihydroxy-6-methyltetrahydro-2H-pyran-2-yl)-D-seryl-L-leucyl-L-arginyl-L- |
| | prolyl)hydrazine-1-carboxamide |

7. Generic Name: Ramorelix

| | |
|---|---|
| a. | Commercial Name: |
| b. | IUPAC Structure: (2S)-2-[[(2R)-2-[[(2S)-2-[[(2S)-2-[[(2R)-2-[[(2R)-2-[[(2R)-2- |
| | acetamido-3-naphthalen-2-ylpropanoyl]amino]-3-(4- |
| | chlorophenyl)propanoyl]amino]-3-(1H-indol-3-yl)propanoyl]amino]-3- |
| | hydroxypropanoyl]amino]-3-(4-hydroxyphenyl)propanoyl]amino]-3- |
| | [(2R,3R,4R,5R,6S)-3,4,5-trihydroxy-6-methyloxan-2-yl]oxypropanoyl]amino]-N- |
| | [(2S)-1-[(2S)-2-[(carbamoylamino)carbamoyl]pyrrolidin-1-yl]-5- |
| | (diaminomethylideneamino)-1-oxopentan-2-yl]-4-methylpentanamide |

8. Generic Name: Antide/Iturelix

| | |
|---|---|
| a. | Commercial Name: |
| b. | IUPAC Structure: N-[(5R)-5-[[(2S)-2-[[(2S)-2-[[(2R)-2-[[(2R)-2-[[(2R)-2- acetamido- |
| | 3-naphthalen-2-ylpropanoyl]amino]-3-(4-chlorophenyl)propanoyl]amino]-3-pyridin- |
| | 3-ylpropanoyl]amino]-3-hydroxypropanoyl]amino]-6-(pyridine-3- |
| | carbonylamino)hexanoyl]amino]-6-[[(2S)-1-[[(2S)-1-[(2S)-2-[[(2R)-1-amino-1- |
| | oxopropan-2-yl]carbamoyl]pyrrolidin-1-yl]-1-oxo-6-(propan-2-ylamino)hexan-2- |
| | yl]amino]-4-methyl-1-oxopentan-2-yl]amino]-6-oxohexyl]pyridine-3-carboxamide |

9. Generic Name: Detirelix/Deterelix

| | |
|---|---|
| a. | Commercial Name: |
| b. | IUPAC Structure: (2S)-1-[(2S)-2-[[(2S)-2-[[(2R)-2-[[(2S)-2-[[(2S)-2-[[(2R)-2-[[(2R) - |
| | 2-[[(2R)-2-acetamido-3-naphthalen-2-ylpropanoyl]amino]-3-(4- |
| | chlorophenyl)propanoyl]amino]-3-(1H-indol-3-yl)propanoyl]amino]-3- |
| | hydroxypropanoyl]amino]-3-(4-hydroxyphenyl)propanoyl]amino]-6- |
| | [bis(ethylamino)methylideneamino]hexanoyl]amino]-4-methylpentanoyl]amino]-5- |
| | carbamimidamidopentanoyl]-N-[(2R)-1-amino-1-oxopropan-2-yl]pyrrolidine-2- |
| | carboxamide |

10. Generic Name: Ozarelix

| | |
|---|---|
| a. | Commercial Name: |
| b. | IUPAC Structure: N-acetyl-3-(2-naphthyl)-D-alanyl-4-chloro-D-phenylalanyl- 3-(3- |
| | pyridyl)-D-alanyl-L-seryl-N-methyl-L-tyrosyl-D-homocitrullyl-L-norleucyl-L-arginyl- |
| | L-prolyl-D-alaninamide |

11. Generic Name: Acyline

| | |
|---|---|
| a. | Commercial Name: |
| b. | IUPAC Structure: N-acetyl-3-(2-naphthyl)-D-alanyl-4-chloro-D-phenylalanyl-3-(3- |
| | pyridyl)-D-alanyl-L-seryl-4-acetamido-L-phenylalanyl-4-acetamido-D-phenylalanyl- |
| | L-leucyl-N6-isopropyl-L-lysyl-L-prolyl-D-alaninamide |

GnRH Antagonists: Non-peptide Small Molecules

1. Generic Name: Elagolix (NBI-42902)

| | |
|---|---|
| a. | Commercial Name: Orlissa |
| b. | IUPAC Structure: 4-[[(1R)-2-[5-(2-fluoro-3-methoxyphenyl)-3-[[2-fluoro-6- |
| | (trifluoromethyl)phenyl]methyl]-4-methyl-2,6-dioxopyrimidin-1-yl]-1- |
| | phenylethyl]amino]butanoic acid |

2. Generic Name: Linzagolix (KLH-21090)

| | |
|---|---|
| a. | Commercial Name: Yselty |
| b. | IUPAC Structure: 3-[5-[(2,3-difluoro-6-methoxyphenyl)methoxy]-2-fluoro- 4- |
| | methoxyphenyl]-2,4-dioxo-1H-thieno[3,4-d]pyrimidine-5-carboxylic acid |

3. Generic Name: Relugolix

| | |
|---|---|
| a. | Commercial Name: Orgovyx, Relumina |
| b. | IUPAC Structure: 1-[4-[1-[(2,6-difluorophenyl)methyl]-5-[(dimethylamino)methyl]- |
| | 3-(6-methoxypyridazin-3-yl)-2,4-dioxothieno[2,3-d]pyrimidin-6-yl]phenyl]-3- |
| | methoxyurea |

4. Generic Name: Opigolix (ASP-17070)

| | |
|---|---|
| a. | Commercial Name: |
| b. | IUPAC Structure: (2R)-N′-[5-[(E)-2-(1H-benzimidazol-2-yl)-3-(2,5-difluorophenyl) - |
| | 1-hydroxy-3-oxoprop-1-enyl]-2-fluorophenyl]sulfonyl-2-hydroxypropanimidamide |

5. Generic Name: Sufugolix (TAK-013)

| | |
|---|---|
| a. | Commercial Name: |
| b. | IUPAC Structure: 1-[4-[5-[[benzyl(methyl)amino]methyl]-1-[(2,6- |
| | difluorophenyl)methyl]-2,4-dioxo-3-phenylthieno[2,3-d]pyrimidin-6-yl]phenyl]-3- |
| | methoxyurea |

6. Generic Name: A-75998

| | |
|---|---|
| a. | Commercial Name: |
| b. | IUPAC Structure: N-[(5R)-5-[[(2S)-2-[[(2S)-2-[[(2R)-2-[[(2R)-2-[[(2R)-2- acetamido- |
| | 3-naphthalen-2-ylpropanoyl]amino]-3-(4-chlorophenyl)propanoyl]amino]-3-pyridin- |
| | 3-ylpropanoyl]amino]-3-hydroxypropanoyl]-methylamino]-3-(4- |
| | hydroxyphenyl)propanoyl]amino]-6-[[(2S)-1-[[(2S)-1-[(2S)-2-[[(2R)-1-amino-1- |
| | oxopropan-2-yl]carbamoyl]pyrrolidin-1-yl]-1-oxo-6-(propan-2-ylamino)hexan-2- |
| | yl]amino]-4-methyl-1-oxopentan-2-yl]amino]-6-oxohexyl]pyridine-3-carboxamide |

Sequences
SEQ ID NO: 1
(human Kisspeptin-10)
YNWNSFGLRF
SEQ ID NO: 2
(Cetrorelix-synthetic peptide)
X1X2X3SYX4LRPX5
Wherein X1 = N-Acetyl-3-(2-naphthyl)-D-alanine;
X2 = 4-Chloro-D-phenylalanine;
X3 = 3-(3-Pyridyl)-D-alanine;
X4 = D-Citrulline; and X5 = D-Alanine

### REFERENCES

Kotani M, Detheux M, Vandenbogaerde A, Communi D, Vanderwinden J M, Le Poul E, et al. The metastasis suppressor gene KiSS-1 encodes kisspeptins, the natural ligands of the orphan G protein-coupled receptor GPR54. J Biol Chem. 2001; 276 (37): 34631-6. doi: 10.1074/jbc.M104847200. PubMed PMID: 11457843.
Smith J T, Li Q, Yap K S, Shahab M, Roseweir A K, Millar R P, et al. Kisspeptin is essential for the full preovulatory LH surge and stimulates GnRH release from the isolated ovine median eminence. Endocrinology. 2011; 152 (3): 1001-12. doi: 10.1210/en.2010-1225. PubMed PMID: 21239443.
Ramaswamy S, Guerriero K A, Gibbs R B, Plant T M. Structural interactions between kisspeptin and GnRH neurons in the mediobasal hypothalamus of the male rhesus monkey (Macaca mulatta) as revealed by double immunofluorescence and confocal microscopy. Endocrinology. 2008; 149 (9): 4387-95. Epub 2008 May 31. doi: 10.1210/en.2008-0438. PubMed PMID: 18511511; PubMed Central PMCID: PMCPMC2553371.
Smith J T, Coolen L M, Kriegsfeld L J, Sari I P, Jaafarzadehshirazi M R, Maltby M, et al. Variation in kisspeptin and RFamide-related peptide (RFRP) expression and terminal connections to gonadotropin-releasing hormone neurons in the brain: a novel medium for seasonal breeding in the sheep. Endocrinology. 2008; 149 (11): 5770-82. Epub 2008 Jul. 12. doi: 10.1210/en.2008-0581. PubMed PMID: 18617612; PubMed Central PMCID: PMCPMC2584593.
Clarkson J, Herbison A E. Postnatal development of kisspeptin neurons in mouse hypothalamus; sexual dimorphism and projections to gonadotropin-releasing hormone neurons. Endocrinology. 2006; 147 (12): 5817-25. Epub 2006 Sep. 9. doi: 10.1210/en.2006-0787. PubMed PMID: 16959837.
Tanco V M, Whitlock B K, Jones M A, Wilborn R R, Brandebourg T D, Foradori C D. Distribution and regulation of gonadotropin-releasing hormone, kisspeptin, RF-amide related peptide-3, and dynorphin in the bovine hypothalamus. PeerJ. 2016; 4: e1833. Epub 2016 Mar. 26. doi: 10.7717/peerj. 1833. PubMed PMID: 27014517; PubMed Central PMCID: PMCPMC4806599.
Caraty A, Smith J T, Lomet D, Ben Said S, Morrissey A, Cognie J, et al. Kisspeptin synchronizes preovulatory surges in cyclical ewes and causes ovulation in seasonally acyclic ewes. Endocrinology. 2007; 148 (11): 5258-67. doi: 10.1210/en.2007-0554. PubMed PMID: 17702853.
Caraty A, Lomet D, Sebert M E, Guillaume D, Beltramo M, Evans N P. Gonadotrophin-releasing hormone release into the hypophyseal portal blood of the ewe mirrors both pulsatile and continuous intravenous infusion of kisspeptin: an insight into kisspeptin's mechanism of action. J Neuroendocrinol. 2013; 25 (6): 537-46. doi: 10.1111/jne. 12030. PubMed PMID: 23387514.
d′Anglemont de Tassigny X, Fagg L A, Carlton M B, Colledge W H. Kisspeptin can stimulate gonadotropin-releasing hormone (GnRH) release by a direct action at GnRH nerve terminals. Endocrinology. 2008; 149 (8): 3926-32. doi: 10.1210/en.2007-1487. PubMed PMID: 18450966; PubMed Central PMCID: PMCPMC2488229.
Ezzat Ahmed A, Saito H, Sawada T, Yaegashi T, Yamashita T, Hirata T, et al. Characteristics of the stimulatory effect of kisspeptin-10 on the secretion of luteinizing hormone, follicle-stimulating hormone and growth hormone in prepubertal male and female cattle. J Reprod Dev. 2009; 55 (6): 650-4. PubMed PMID: 19789422.
Matsui H, Takatsu Y, Kumano S, Matsumoto H, Ohtaki T. Peripheral administration of metastin induces marked gonadotropin release and ovulation in the rat. Biochem Biophys Res Commun. 2004; 320 (2): 383-8. doi: 10.1016/j.bbrc.2004.05.185. PubMed PMID: 15219839.
Leonardi C E P, Dias F C F, Adams G P, Araujo E R, Singh J. Kisspeptin induces ovulation in heifers under low plasma progesterone concentrations. Theriogenology. 2020; 141:26-34. Epub 2019 Sep. 9. doi: 10.1016/j.theriogenology.2019.08.033. PubMed PMID: 31494459.
Leonardi C E P, Dias F C F, Adams G P, Singh J. Effect of Kisspeptin-10 on plasma luteinizing hormone concentrations and follicular dynamics during the luteal phase in cattle. Theriogenology. 2018; 119:268-74. Epub 2018 Aug. 3. doi: 10.1016/j.theriogenology.2018.06.023. PubMed PMID: 30071491.
Vizcarra J A, Wettemann R P, Braden T D, Turzillo A M, Nett T M. Effect of gonadotropin-releasing hormone (GnRH) pulse frequency on serum and pituitary concentrations of luteinizing hormone and follicle-stimulating hormone, GnRH receptors, and messenger ribonucleic acid for gonadotropin subunits in cows. Endocrinology. 1997; 138 (2): 594-601. doi: DOI 10.1210/en. 138.2.594. PubMed PMID: WOS: A1997WC64600012.
Yoshioka K, Suzuki C, Arai S, Iwamura S, Hirose H. Gonadotropin-releasing hormone in third ventricular cerebrospinal fluid of the heifer during the estrous cycle. Biol Reprod. 2001; 64 (2): 563-70. PubMed PMID: 11159359.
Ginther O J, Santos V G, Mir R A, Beg M A. Role of L H in the progesterone increase during the bromocriptine-induced prolactin decrease in heifers. Theriogenology. 2012; 78 (9): 1969-76. doi: 10.1016/j.theriogenology.2012.08.003. PubMed PMID: WOS: 000310858700010.
Whitlock B K, Daniel J A, Wilborn R R, Rodning S P, Maxwell H S, Steele B P, et al. Interaction of estrogen and progesterone on kisspeptin-10-stimulated luteinizing hormone and growth hormone in ovariectomized cows. Neuroendocrinology. 2008; 88 (3): 212-5. doi: 10.1159/000146242. PubMed PMID: 18635924.
Kadokawa H, Matsui M, Hayashi K, Matsunaga N, Kawashima C, Shimizu T, et al. Peripheral administration of kisspeptin-10 increases plasma concentrations of GH as well as LH in prepubertal Holstein heifers. J Endocrinol. 2008; 196 (2): 331-4. doi: 10.1677/JOE-07-0504. PubMed PMID: 18252956.
Ezzat A A, Saito H, Sawada T, Yaegashi T, Goto Y, Nakajima Y, et al. The role of sexual steroid hormones in the direct stimulation by Kisspeptin-10 of the secretion of luteinizing hormone, follicle-stimulating hormone and prolactin from bovine anterior pituitary cells. Anim Reprod Sci. 2010; 121 (3-4): 267-72. Epub 2010 Jul. 3. doi: 10.1016/j.anireprosci.2010.06.002. PubMed PMID: 20594780.
Magee C, Bruemmer J E, Kirkley K S, Sylvester L A, Runyan B, Nett T M, et al. Kisspeptin has an independent and direct effect on the pituitary gland in the mare. Theriogenology. 2020; 157:199-209. Epub 2020 Aug. 20. doi: 10.1016/j.theriogenology.2020.07.031. PubMed PMID: 32814247.
Bergfelt D R, Smith C A, Adams G P, Ginther O J. Surges of FSH during the follicular and early luteal phases of the estrous cycle in heifers. Theriogenology. 1997; 48 (5): 757-68. PubMed PMID: 16728169.
Bergfelt D R, Lightfoot K C, Adams G P. Ovarian Synchronization Following Ultrasound-Guided Transvaginal Follicle Ablation in Heifers. Theriogenology. 1994; 42 (6): 895-907. PubMed PMID: WOS: A1994PP55800001.
Bolt D J, Scott V, Kiracofe G H. Plasma-Lh and Fsh after Estradiol, Norgestomet and Gn-Rh Treatment in Ovariectomized Beef Heifers. Animal Reproduction Science. 1990; 23 (4): 263-71. doi: Doi 10.1016/0378-4320 (90) 90040-M. PubMed PMID: WOS: A1990EM89300001.
Ginther O J, Bergfelt D R, Kulick L J, Kot K. Selection of the dominant follicle in cattle: Establishment of follicle deviation in less than 8 hours through depression of FSH concentrations. Theriogenology. 1999; 52 (6): 1079-93. doi: Doi 10.1016/S0093-691x(99) 00196-X. PubMed PMID: WOS: 000083806400012.
Ginther O J, Beg M A, Gastal E L, Gastal M O, Baerwald A R, Pierson R A. Systemic concentrations of hormones during the development of follicular waves in mares and women: a comparative study. Reproduction. 2005; 130 (3): 379-88. doi: 10.1530/rep.1.00757. PubMed PMID: WOS: 000231946900014.
Ginther O J, Khan F A, Hannan M A, Rodriguez M B, Pugliesi G, Beg M A. Role of LH in luteolysis and growth of the ovulatory follicle and estradiol regulation of LH secretion in heifers. 35 Theriogenology. 2012; 77 (7): 1442-52. Epub 2012 Jan. 31. doi: 10.1016/j.theriogenology.2011.11.014. PubMed PMID: 22284221.
Oussaid B, Lonergan P, Khatir H, Guler A, Monniaux D, Touze J L, Beckers J F, Cognie Y, Mermillod P. Effect of GnRH antagonist-induced prolonged follicular phase on follicular atresia and oocyte developmental competence in vitro in superovulated heifers. J Reprod Fertil. 2000 January; 118 (1): 137-44.
Madill S, Rieger D, Johnson W H, Walton J S, Coy D H, Rawlings N C. Effects of an LHRH antagonist on the time of occurrence and amplitude of the preovulatory LH surge, progesterone and estradiol secretion, and ovulation in superovulated Holstein heifers. Theriogenology. 1994; 41 (4): 951-60.
Fike K E, Bergfeld E G, Cupp A S, Kojima F N, Mariscal V, Sanchez T, Wehrman M E, Grotjan W H, Hamernik D L, Kittok R J, Kinder J E. Gonadotropin secretion and development of ovarian follicles during oestrous cycles in heifers treated with luteinizing hormone releasing hormone antagonist. Anim Reprod Sci. 1997 Dec. 5; 49 (2-3): 83-100.
Haughian J M, Ginther O J, Diaz F J, Wiltbank M C. Gonadotropin-releasing hormone, estradiol, and inhibin regulation of follicle-stimulating hormone and luteinizing hormone surges: implications for follicle emergence and selection in heifers. Biol Reprod. 2013 Jun. 27; 88 (6): 165.
Adams G P, Sumar J, Ginther O J. 1990. Effects of lactational and reproductive status on ovarian follicular waves in llamas (Lama glama). J Reprod Fertil 90, 535-545.
Adams, G. P., Matteri, R. L., Kastelic, J. P., Ko, J. C., Ginther, O. J., 1992. Association between surges of follicle-stimulating hormone and the emergence of follicular waves in heifers. Journal of reproduction and fertility 94, 177-188.
Adam C L, Bourke D A, Kyle C E, Young P, McEvoy T G. 1992. Ovulation and embryo recovery in the llama. In: Allen W R, Higgins A J, Mayhew I G, Snow D, Wade J F (eds): Proceedings of the first International Camel Conference. R & W Publications, Newmarket, U K, 125-127.
Adams G P, Jaiswal R, Singh J, Malhi P (2008) Progress in understanding ovarian follicular dynamics in cattle. Theriogenology 69:72-80.
Adams, G., Singh, J., 2021. Ovarian follicular and luteal dynamics in cattle, In: Hopper, R. M. (Ed.), Bovine reproduction, Wiley Blackwell, Hoboken, NJ, USA, pp. 292-323.
Aller J F, Cancino A K, Rebuffi G, Alberio R H. 1999. Induccion de la ovulacion en llamas. Libro de Resumenes II congreso Mundial sobre Camelidos, Cusco, Peru, 91. Abstr.
Bravo P W, Stabenfeldt G H, Fowler M E, Lasley B L. 1992. Pituitary response to repeated copulation and/or gonadotropin-releasing hormone administration in llamas and alpacas. Biol Reprod 47, 884-888.
Bravo P W, Stabenfeldt G H, Lasley B L, Fowler M E. 1991. The effect of ovarian follicular size on pituitary and ovarian responses to copulation in domesticated South American camelids. Biol Reprod 45, 553-559.
Cancino A K, Aller J F, Rebuffi G, Alberio R H. 1999. El uso de norgestomet para la sincronizacion de la onda folicular en llamas en dos epocas del ano (invierno y verano). Libro de Resumenes I I Congreso Mundial sobre Camelidos, Cusco, Peru, 93. Abstr.
Correa J E, Ratto M H, Gatica R. 1997. Superovulation in llamas (Lama glama) with pFSH and equine chorionic gonadotropin used individually or in combination. Anim Reprod Sci 46, 289-296.
EMD Serono Physician Package Insert (2008) Cetrorelix acetate for injection. EMD Serono Inc., p1-10.
England B G, Foot W C, Matthews D H, Cardozo A G, Riera S. 1969. Ovulation and corpus luteum function in the llama (Lama glama). J Endocr 45, 505-513.
Fernandez-Baca S, Madden D H L, Novoa C. 1970a. Effect of different mating stimuli on induction of ovulation in the alpaca. J Reprod Fertil 22, 261-267.
Ginther, O. J., Kastelic, J. P., Knopf, L., 1989. Composition and characteristics of follicular waves during the bovine estrous cycle. Animal reproduction science 20, 187-200.
Huanca W, Cardenas O, Olazabal C, Ratto M, Adams G P. 2001. Efecto hormonal y empadre sobre el intervalo a la ovulacion en llamas. Revista de Investigaciones Veterinarias del Peru 1, 462-463.
Knopf, L., Kastelic, J. P., Schallenberger, E., Ginther, O. J., 1989. Ovarian follicular dynamics in heifers: test of two-wave hypothesis by ultrasonically monitoring individual follicles. Domest Anim Endocrinol 6, 111-119.
Kovacs M, Schally A V, Csernus B, Rekasi Z (2001) Luteinizing hormone-releasing hormone (LH-RH) antagonist Cetrorelix down-regulates the mRNA expression of pituitary receptors for LHRH by counteracting the stimulatory effect of endogenous LH-RH. PNAS 98:1829-1834.
Leonardi C E (2018) Kisspeptin function in female bovine reproduction. Doctoral Thesis Dissertation, University of Saskatchewan (https://harvest.usask.ca/handle/10388/9597).
Nivet A L, Vigneault C, Blondin P, Sirard M A (2018) Influence of luteinizing hormone support on granulosa cells transcriptome in cattle. Animal Science Journal 89:21-30.
Ratto M H, Gatica R, Correa J E. 1997. Timing of mating and ovarian response in llamas (Lama glama) treated with pFSH. Anim Reprod Sci 48, 325-330.
San Martin M, Copaira M, Zuniga J, Rodriguez R, Bustinza G, Acosta L. Aspects of reproduction in the alpaca. 1968. J Reprod Fertil 16, 395-399.
Silva M E, Smulders J P, Guerra M, Valderrama X P, Letelier C, Adams G P, Ratto M H. Cetrorelix suppresses the preovulatory LH surge and ovulation induced by ovulation-inducing factor (OIF) present in llama seminal plasma. Reprod Biol Endocrinol. 2011 May 30; 9:74.
Taylor S, Taylor P J, James A N, Godke R A. 2000. Successful commercial embryo transfer in the llama (Lama glama). Theriogenoloy 53, 344. Abst.
Ulker H, Gant B T, de Avila D M, Reeves J J. LHRH antagonist decreases LH and progesterone secretion but does not alter length of estrous cycle in heifers. J Anim Sci. 2001 November; 79 (11): 2902-7.
Vaughan J, Macmillan K L, D'Occhio M J. 2004. Ovarian follicular wave characteristics in alpacas. Anim Reprod Sci 80, 353-361.

1. (canceled)

2. A non-therapeutic method of synchronizing ovulation in a population of female mammals, the method comprising:
I)
a) synchronizing follicular wave emergence (FWE) by administering an effective amount of a GnRH antagonist; and
b) administering an effective amount of an ovulation inducing agent at a fixed time following the administration of the GnRH antagonist, to each mammal of the population of female mammals; or
II)
a) synchronizing follicular wave emergence (FWE) by administering an effective amount of a GnRH antagonist and an effective amount of progesterone or a progesterone analog;
b) withdrawing administration of the progesterone or progesterone analog and administering an effective amount of prostaglandin (PGF) or a PGF analog at a fixed time following the administration of the GnRH antagonist; and
c) administering an effective amount of an ovulation inducing agent at a fixed time following the withdrawal of progesterone or progesterone analog and/or administration of the PGF or PGF analog, to each mammal of the population of female mammals.

3. The method of claim 2, wherein the GnRH antagonist comprises Cetrorelix, acyline, antarelix/teverelix, degarelix, ganirelix, antide, relugolix, elagolix, abarelix, prazarelix, ramorelix, antide, detirelix, ozarelix, linzagolix, opigolix, sufugolix or A-75998.

4-8. (canceled)

9. The method of claim 2, wherein:
the administering the GnRH antagonist comprises at least one treatment, optionally 2 or 3 treatments; or
the GnRH antagonist comprises Cetrorelix delivered by intramuscular or subcutaneous injection, optionally at a dose of about 5 ug/kg body weight to about 200 ug/kg body weight, optionally at a dose of about 20 ug/kg body weight or about 40 ug/kg body weight for intramuscular injection.

10. (canceled)

11. The method of claim 2, wherein
the GnRH antagonist is administered by vaginal drug releasing device; and/or
the progesterone or progesterone analog is administered by vaginal drug releasing device.

12. (canceled)

13. The method of claim 2, wherein
the mammals are domestic livestock, optionally the domestic livestock are bovine, camelid, equine, porcine, ovine or caprine; or
the mammals are wildlife animals, optionally bison, elk, caribou, deer, muskox, non-human primates, canines, or felines, optionally tigers, lions, or panthers.

14. (canceled)

15. The method of claim 2, wherein the population is a mixed population.

16. The method of claim 2, further comprising
i) artificially inseminating each mammal of the population of female mammals at a fixed time following administration of the GnRH antagonist, and optionally collecting one or more embryos from each mammal of the population of female mammals at a fixed time following artificial insemination;
ii) mating each mammal of the population of female mammals at a fixed time following administration of the GnRH antagonist, and optionally collecting one or more embryos from each mammal of the population of female mammals at a fixed time following mating; or
iii) transferring an embryo to each animal of the population of female mammals at a fixed time following administration of the GnRH antagonist.

17-28. (canceled)

29. A non-therapeutic method of inducing ovulation in a female mammal, the method comprising:
I)
a) initiating follicular wave emergence (FWE) by administering an effective amount of a GnRH antagonist to the female mammal; and
b) administering an effective amount of an ovulation inducing agent at a fixed time following the administration of the GnRH antagonist; or
II)
a) initiating follicular wave emergence (FWE) by administering an effective amount of a GnRH antagonist to the female mammal;
b) co-administering an effective amount of progesterone or a progesterone analog with the GnRH antagonist;
c) withdrawing the progesterone or progesterone analog and administering an effective amount of prostaglandin (PGF) or a PGF analog at a fixed time following the administration of the GnRH antagonist; and
d) administering an effective amount of an ovulation inducing agent at a fixed time following the withdrawal of the progesterone or progesterone analog and/or administration of the PGF or PGF analog.

30. A non-therapeutic fixed-time method comprising inducing ovulation according to the method of claim 29; and further comprising:
A) artificially inseminating the mammal at a fixed time following the administration of the ovulation inducing agent; optionally further comprising collecting one or more embryos at a fixed time following artificial insemination;
B) mating the mammal at a fixed time following the administration of the ovulation inducing agent; optionally further comprising collecting one or more embryos at a fixed time following mating; or
C) transferring one or more embryos to the female mammal at a fixed time following the administration of the ovulation inducing agent.

31-32. (canceled)

33. A non-therapeutic fixed-time superstimulatory or superovulatory method, the method comprising:
I)
a) administering an effective amount of a GnRH antagonist to a female mammal; and b) administering an effective amount of FSH or an FSH agonist to the female mammal at a fixed time following the administration of the GnRH antagonist, provided that the FSH or FSH agonist is not co-administered with the GnRH antagonist, optionally further comprising administering an effective amount of an ovulation inducing agent at a fixed time following the administration of the FSH or FSH agonist; or
II)
a) administering an effective amount of a GnRH antagonist to a female mammal; b) co-administering an effective amount of progesterone or a progesterone analog with the GnRH antagonist; c) administering an effective amount of FSH or an FSH agonist to the female mammal at a fixed time following the administration of the GnRH antagonist, provided that the FSH or FSH agonist is not co-administered with the GnRH antagonist; and d) withdrawing the progesterone or progesterone analog and administering an effective amount of prostaglandin (PGF) or a PGF analog at a fixed time following the administration of the FSH or FSH agonist, optionally further comprising administering an effective amount of an ovulation inducing agent at a fixed time following the withdrawal of the progesterone or progesterone analog and/or administration of the PGF or PGF analog.

34. (canceled)

35. The non-therapeutic fixed-time method of claim 33, further comprising:
collecting one or more oocytes at a fixed time following the administration of the GnRH antagonist, the FSH, the PGF, or the ovulation inducing agent of II).

36-37. (canceled)

38. The method of claim 29, wherein the PGF or PGF analog comprises PGF2a.

39. The method of claim 29, wherein the ovulation inducing agent comprises GnRH or a GnRH analog or agonist, LH or an LH analog, agonist, conjugate or recombinant product, or estradiol or an estradiol ester, analog, or agonist.

40. The method of claim 29, wherein
the mammal is a domestic livestock animal, optionally the domestic livestock animal is bovine, camelid, equine, porcine, ovine, or caprine;
the mammal is a wildlife animal, optionally bison, elk, caribou, deer, muskox, non-human primate, canine, or feline; or
the mammal is a human.

41-42. (canceled)

43. The method of claim 29, wherein the progesterone or progesterone analog is administered by vaginal drug releasing device, optionally wherein the progesterone or progesterone analog drug releasing device is administered for 7-8 days when the mammal is a bovine.

44-45. (canceled)

46. The method of claim 43, wherein the GnRH antagonist is administered on day 0 (D0), and the method further comprises i) inserting the progesterone or progesterone analog drug releasing device on D0; ii) administering an effective amount of prostaglandin (PGF2a) or prostaglandin analog and removing the progesterone or progesterone analog drug releasing device on day 8 (D8); and iii) administering an effective amount of GnRH on day 10 (D10).

47-52. (canceled)

53. The method of claim 29, wherein the GnRH antagonist comprises Cetrorelix, abarelix, degarelix, ganirelix, antarelix/teverelix, prazarelix, ramorelix, antide, detirelix, ozarelix, acyline, elagolix, linzagolix, relugolix, opigolix, sufugolix or A-75998.

54. The method of claim 29, wherein
the GnRH antagonist is administered by intramuscular injection or subcutaneous injection; and/or
the GnRH antagonist and/or the progesterone or progesterone analog is administered by vaginal drug releasing device.

55. The method of claim 29, wherein the administering the GnRH antagonist comprises at least one treatment, optionally 2 or 3 treatments.

56. The method of claim 29, wherein the GnRH antagonist comprises Cetrorelix delivered by intramuscular injection at a dose of about 20 ug/kg body weight to about 40 ug/kg body weight.

57. The method of claim 2, wherein the PGF or PGF analog comprises PGF2a.

58. The method of claim 2, wherein the ovulation inducing agent comprises GnRH or a GnRH analog or agonist, LH or an LH analog, agonist, conjugate or recombinant product, or estradiol or an estradiol ester, analog, or agonist.
